(19) United States
(12) Patent Application Publication
(10) Pub. No.: US 2022/0064104 A1
(43) Pub. Date: Mar. 3, 2022

(54) AN ALL-OPTICAL EXCITONIC SWITCH OPERATED IN LIQUID AND SOLID PHASES

(71) Applicants: Boise State University, Boise, ID (US); Heidelberg University, Heidelberg (DE)

(72) Inventors: William B. Knowlton, Boise, ID (US); Bernard Yurke, Boise, ID (US); Donald L. Kellis, Boise, ID (US); Paul H. Davis, Boise, ID (US); Elton Graugnard, Boise, ID (US); Jeunghoon Lee, Boise, ID (US); Brittany L. Cannon, Boise, ID (US); Andres Jäschke, Heidelberg (DE); Christopher Sarter, Heidelberg (DE)

(21) Appl. No.: 17/310,335

(22) PCT Filed: Jan. 28, 2020
(86) PCT No.: PCT/US20/15382

§ 371 Date: Jul. 28, 2021

Related U.S. Application Data
(60) Provisional application No. 62/797,786, filed on Jan. 28, 2019.

(51) Int. Cl. C07C 229/18; G01N 21/64; C12Q 1/68

(52) U.S. Cl. CPC C07C 229/18; C12Q 1/68; G01N 21/64

(57) ABSTRACT

The present disclosure is directed to an all-optical excitonic switch comprising one or two oligonucleotides that comprises in turn donor/acceptor chromophores and photochromic nucleotide and is assembled with nanometer scale precision using DNA nanotechnology. The disclosed all-optical excitonic switches operate successfully in both liquid and solid phases, exhibiting high ON/OFF switching contrast with no apparent cyclic fatigue. The all-optical excitonic switches disclosed herein have small footprint and volume, low energy requirement, and potential ability to switch at speeds in tens of picosecond.

### CROSS-REFERENCE TO RELATED APPLICATIONS

This application claims priority to Provisional Application U.S. Ser. No. 62/797,786 filed on Jan. 28, 2019, all of which is herein incorporated by reference in its entirety.

### FIELD OF THE INVENTION

The present disclosure relates generally to the field of all-optical excitonic circuitry. In particular, the present disclosure relates to an all-optical excitonic switch comprising donor/acceptor chromophores and photochromic nucleotide and assembled with nanometer scale precision using DNA nanotechnology. The disclosed all-optical excitonic switches operate successfully in both liquid and solid phases, exhibiting high ON/OFF switching contrast with no apparent cyclic fatigue through nearly 200 cycles. The all-optical excitonic switches disclosed herein have small footprint and volume, low energy requirement, and potential ability to switch at speeds in tens of picoseconds.

### BACKGROUND OF THE INVENTION

With the semiconductor industry rapidly approaching the end of Moore's law, excitonics offers an attractive alternative to semiconductor electronics for information processing. Excitonics exploits spatially proximate optically functional components to capture and transport light energy below the diffraction limit of light.

Photosynthetic organisms in nature, which possess exquisite excitonic networks such as light harvesting systems that are >100 times smaller than the wavelength of visible light, provide insight and inspiration to design an all-optical excitonic switch. Challenges to constructing technologically relevant excitonic circuits include identifying suitable: (i) substrates or scaffolds on which optically active components can be arranged into complex and scalable networks with nanometer scale precision and control, and (ii) device architectures for gates that employ only excitons in their operations.

Despite these challenges, natural light harvesting and photovoltaics, as well as excitonic devices and logic gates, were developed. Examples of such devices include light harvesting systems employ proteins as a scaffold to self-assemble light active molecules or chromophores into excitonic networks that operate at room temperature in wet and noisy environments. In these devices, the packing density of the chromophores is much higher than the component density of the most advanced semiconductor electronic circuits, and exciton transport times rival electronic gate switching times.

Self-assembly with proteins, however, is very complicated owing to their over twenty amino acid protein building blocks and the difficulty of predicting apriori how peptide sequences will fold. In contrast, with only four nucleic acid building blocks and well-established design rules, DNA offers a more rational and feasible method of programmable self-assembly with elegant control that yields exquisite one-, two-, and three-dimensional nanostructures.

Various FRET-based excitonic devices, typically with donor-acceptor dye pairs self-assembled onto static or dynamic DNA scaffolds, have also been developed. These include switches, logic gates, and energy ratchet switches. However, the switching processes employed in these devices require aqueous solutions with chemical compositions that enable DNA duplex dissociation via DNA strand invasion, DNA-intercalation, or chromophore modulation. Furthermore, these switching processes have rate-limiting steps such as strand displacement, mass transport, and reducing agent diffusion which lead to challenges including cyclic fatigue and long cycle times. Although excitonic switching performed in a liquid phase may be useful in super resolution imaging or medical diagnostics, an all-optical switch operating in a solid or liquid phase is preferred to create a viable excitonics technology.

In other words, while existing all-optical excitonic switches offer compelling examples of potential applications in excitonic circuit-based information processing and demonstrate the possibility of using a photochromic moiety to modulate covalently attached donors in a process termed photochromic Förster resonance energy transfer (pcFRET), an all-optical exitonic switch in which all-optical components are arranged in complex, well-defined, and scalable networks have yet to be achieved.

Accordingly, it is an objective of the present disclosure to disclose an all-optical excitonic switch.

These and other objects, advantages and features of the present disclosure will become apparent from the following specification taken in conjunction with the claims set forth herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a general scheme for the synthesis of an exemplary phosphoramidite for solid phase synthesis.

FIG. 2 shows the HPLC chromatograms of the synthesized modified oligonucleotides with one modification (PS1) and three modifications (PS3), respectively, detected at 280 nm.

FIG. 3A shows the normalized absorption and emission spectra of all chromophores present in the all-optical excitonic switch.

FIG. 3B shows the actual unaltered absorbance of the acceptor as well as the three photochromic nucleotides in their open and closed states.

FIG. 4A-FIG. 4H show an exemplary all-optical excitonic switch and its characteristics.

FIG. 5A-FIG. 5D show saw-tooth plots of the all-optical excitonic switch operating in the liquid phase.

FIG. 6 shows the detector geometry for acquisition of all-optical excitonic emission spectra.

FIG. 7A-FIG. 7L show all-optical excitonic switch controls performed at a 5 μM DNA concentration in the liquid environment.

FIG. 8A-FIG. 8F show the switch emission of an exemplary all-optical excitonic switch with three photochromic nucleotides attached.

FIG. 9A-FIG. 9D show the representative solid phase sample imaged using a 534 nm excitation filter and a 606 nm emission filter, 3D profilometer map thereof, and profilometer data thereof.

FIG. 10A-FIG. 10F show the characteristic all-optical excitonic switch time.

FIG. 11A-FIG. 11B show the cyclic fatigue assessment of an exemplary all-optical switch.

FIG. 12 shows the HPLC time trace of PS1 after 10 min of UV-irradiation with a 310 nm LED (Thorlabs M310L3) at 260 nm.

### BRIEF SUMMARY OF THE INVENTION

Disclosed herein is an all-optical switch comprising DNA oligonucleotides covalently integrated with photochromic units, such as azobenzenes and diarylethenes.

In one aspect, disclosed herein is an all-optical excitonic switch, the switch comprises one, two, or more oligonucleotides, wherein at least one oligonucleotide comprises one or more photochromic nucleotides; at least one oligonucleotide comprises a donor, and at least one oligonucleotide comprises an acceptor, wherein the photochromic nucleotide can undergo reversible photoisomerization under different wavelengths; an excitonic transfer occurs between emission of the donor and absorption of the acceptor; and the emission of the donor and the absorption of the acceptor fall within absorption of the one or more photochromic nucleotides.

In another aspect, disclosed herein is a method for creating an “ON” or “OFF” state, wherein the method comprising illuminating with a first light and then second light to the all-optical switch disclosed herein, wherein the all-optical switch emits an emission light signal and the emission light signal varies depending on if the first light and second light are on at the same time.

In yet another aspect, disclosed herein is a device for data processing or storing information, wherein the device comprises one or more all-optical switches disclosed herein and wherein the all-optical switches function as flash memory or transistor.

In some embodiments, the device is a flash memory device. In some other embodiments, the device is a digital processor.

The all-optical excitonic switches disclosed herein utilize photoisomerization, one of the fastest reactions studied to date. The all-optical excitonic switches can be assembled by first integrating a photochromic unit nucleobase into a nucleobase, which can pair with its complementary nucleotide, and then synthesizing through automated solid-phase phosphoramidite chemistry a DNA oligonucleotide containing multiple photochromic units along the strand length.

The all-optical excitonic switches disclosed herein can operate in both liquid and solid phase. An exemplary all-optical excitonic switch comprising a DNA duplex demonstrate exceptional switching contrast between the ON and OFF states with no evidence of cyclic fatigue and at sub-nanosecond speeds. Because the all-optical excitonic switches disclosed herein are based on DNA duplexes, the switches are easily scalable and programmable. The switches disclosed herein are significantly smaller and faster than current electronic circuits.

The forgoing summary is illustrative only and is not intended to be in any way limiting. In addition to the illustrative aspects, embodiments and features described above, further aspects, embodiments, and features of the present technology will become apparent to those skilled in the art from the following drawings and the detailed description, which shows and describes illustrative embodiments of the present technology. Accordingly, the figures and detailed description are also to be regarded as illustrative in nature and not in any way limiting.

### DETAILED DESCRIPTION OF THE PREFERRED EMBODIMENT

In the following detailed description, reference may made to the accompanying drawings, schemes, and structures which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The embodiments of this disclosure are not limited to any specific compositions and methods which can vary and are understood by skilled artisans. It is further to be understood that all terminology used herein is for describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (i.e. 1 to 5 includes 1, 1.5, 2, 2.75, 3, 3.80, 4, and 5).

So that the present disclosure may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and liquid handling procedures used for making concentrates or use solutions in the real world; through error in these procedures; through differences in the manufacture, source, or purity of the ingredients used to make the compositions or carry out the methods; and the like. The term “about” also encompasses amounts that differ due to novel equilibrium conditions for a composition resulting from a particular initial mixture. Whether or not modified by the term “about”, the claims include equivalents to the quantities.

As used herein, “substituted” refers to an organic group as defined below (i.e., an alkyl group) in which one or more bonds to a hydrogen atom contained therein are replaced by a bond to non-hydrogen or non-carbon atoms. Substituted groups also include groups in which one or more bonds to carbon(s) or hydrogen(s) atom replaced by one or more bonds, including double or triple bonds, to a heteroatom. Thus, a substituted group is substituted with one or more substituents, unless otherwise specified. A substituted group can be substituted with 1, 2, 3, 4, 5, or 6 substituents.

Substituted ring groups include rings and ring systems in which a bond to a hydrogen atom is replaced with a bond to a carbon atom. Therefore, substituted cycloalkyl, aryl, heterocyclyl, and heteroaryl groups may also be substituted with substituted or unsubstituted alkyl, alkenyl, and alkynyl groups are defined herein.

As used herein, the term “alkyl” or “alkyl groups” refers to saturated hydrocarbons having one or more carbon atoms, including straight-chain alkyl groups (i.e., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (i.e., cyclopropyl, cyclopentyl, cyclohexyl, cycloheptyl, cyclooctyl, etc.), branched-chain alkyl groups (i.e., isopropyl, tert-butyl, sec-butyl, isobutyl, etc.), and alkyl-substituted alkyl groups (i.e., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Alkenyl groups or alkenes are straight chain, branched, or cyclic alkyl groups having two to about 30 carbon atoms, and further including at least one double bond. In some embodiments, an alkenyl group has from 2 to about 30 carbon atoms, or typically, from 2 to 10 carbon atoms. Alkenyl groups may be substituted or unsubstituted. For a double bond in an alkenyl group, the configuration for the double bond can be a trans or cis configuration. Alkenyl groups may be substituted similarly to alkyl groups.

Alkynyl groups are straight chain, branched, or cyclic alkyl groups having two to about 30 carbon atoms, and further including at least one triple bond. In some embodiments, an alkynyl group has from 2 to about 30 carbon atoms, or typically, from 2 to 10 carbon atoms. Alkynyl groups may be substituted or unsubstituted. Alkynyl groups may be substituted similarly to alkyl or alkenyl groups.

As used herein, the terms “alkylene”, “cycloalkylene”, “alkynylides”, and “alkenylene”, alone or as part of another substituent, refer to a divalent radical derived from an alkyl, cycloalkyl, or alkenyl group, respectively, as exemplified by —CH₂CH₂CH₂—. For alkylene, cycloalkylene, alkynylene, and alkenylene groups, no orientation of the linking group is implied.

The term “ester” as used herein refers to —R³⁰COOR³¹ group. R³⁰ is absent, a substituted or unsubstituted alkylene, cycloalkylene, alkenylene, alkynylene, arylene, aralkylene, heterocyclylalkylene, or heterocyclylene group as defined herein. R³¹ is a substituted or unsubstituted alkyl, cycloalkyl, alkenyl, alkynyl, aryl, aralkyl, heterocyclylalkyl, or heterocyclyl group as defined herein.

The term “amine” (or “amino”) as used herein refers to —R³²NR³³R³⁴ groups. R³² is absent, a substituted or unsubstituted alkylene, cycloalkylene, alkenylene, alkynylene, arylene, aralkylene, heterocyclylalkylene, or heterocyclylene group as defined herein. R³³ and R³⁴ are independently hydrogen, or a substituted or unsubstituted alkyl, cycloalkyl, alkenyl, alkynyl, aryl, aralkyl, heterocyclylalkyl, or heterocyclyl group as defined herein.

The term “amine” as used herein also refers to an independent compound. When an amine is a compound, it can be represented by a formula of R^{32′}NR^{33′}R^{34′} groups, wherein R^{32′}, R^{33′}, and R³⁴ are independently hydrogen, or a substituted or unsubstituted alkyl, cycloalkyl, alkenyl, alkynyl, aryl, aralkyl, heterocyclylalkyl, or heterocyclyl group as defined herein.

The term “alcohol” as used herein refers to —R³⁵OH groups. R³⁵ is absent, a substituted or unsubstituted alkylene, cycloalkylene, alkenylene, alkynylene, arylene, aralkylene, heterocyclylalkylene, or heterocyclylene group as defined herein.

The term “carboxylic acid” as used herein refers to —R³⁶COOH groups. R³⁶ is absent, a substituted or unsubstituted alkylene, cycloalkylene, alkenylene, alkynylene, arylene, aralkylene, heterocyclylalkylene, or heterocyclylene group as defined herein.

The term “ether” as used herein refers to —R³⁷OR³⁸ groups. R³⁷ is absent, a substituted or unsubstituted alkylene, cycloalkylene, alkenylene, alkynylene, arylene, aralkylene, heterocyclylalkylene, or heterocyclylene group as defined herein. R³⁸ is a substituted or unsubstituted alkyl, cycloalkyl, alkenyl, alkynyl, aryl, aralkyl, heterocyclylalkyl, or heterocyclyl group as defined herein.

Disclosed herein are all-optical switches comprising one or more oligonucleotides, wherein at least one oligonucleotide comprises one or more photochromic nucleotides; at least one oligonucleotide comprises a donor, and at least one oligonucleotide comprises an acceptor, wherein photochromic nucleotide can undergo reversible photoisomerization under different wavelengths; an excitonic transfer occurs between emission of the donor and absorption of the acceptor; and the emission of the donor and the absorption of the acceptor fall within absorption of the one or more photochromic nucleotides.

### Oligonucleotides

The all-optical switches disclosed herein are one, two or more oligonucleotides, at least one of which comprises a donor, acceptor, and one or more photochromic nucleotides. As used herein, an oligonucleotide can contain all the natural nucleotides found in nature or one, more, or all modified or synthetic nucleotides, in addition to the natural nucleotides and the nucleotides containing the donor, acceptor, or photochromic moiety. A modified or synthetic nucleotide in the oligonucleotides can differ from a natural occurring nucleotide in its base, sugar, and/or backbone moiety.

The oligonucleotide in the all-optical switches disclosed herein can be, but are not limited to, a peptide nucleic acid (PNA), a locked nucleic acid (LNA), a bridged nucleic acid polymer, or combination thereof.

PNA is an artificially synthesized polymer like DNA or RNA. While DNA and RNA have a deoxyribose and ribose sugar backbone, respectively, PNA's backbone is composed of repeating N-(2-aminoethyl)-glycine units linked by peptide bonds. The various purine and pyrimidine bases are linked to the backbone by a methylene bridge (—CH₂—) and a carbonyl group (—(C═O)—).

PNA oligomers can show greater specificity in binding to complementary DNAs, with a PNA/DNA base mismatch being more destabilizing than a similar mismatch in a DNA/DNA duplex.

A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. LNA polymer are synthesized chemically and are commercially available. The locked ribose conformation enhances base stacking and backbone pre-organization. This significantly increases the hybridization properties (melting temperature) of oligonucleotides.

Bridged nucleic acids (BNAs) are modified RNA nucleotides. They are sometimes also referred to as constrained or inaccessible RNA molecules. BNA monomers can contain a five-membered, six-membered, or even a seven-membered bridged structure with a “fixed” C3′-endo sugar puckering. The bridge is synthetically incorporated at the 2′, 4′-position of the ribose to afford a 2′, 4′-BNA monomer. The monomers can be incorporated into oligonucleotide polymeric structures using standard phosphoamidite chemistry. BNAs are structurally rigid oligo-nucleotides with increased binding affinities and stability.

The oligonucleotide as used herein can be a DNA strand containing mainly natural or modified A, T, C, G nucleotide, and/or derivative thereof. The oligonucleotide can also be RNA strand containing mainly natural or modified A, U, C, G nucleotide, and/or derivative thereof. The oligonucleotide can be a mixed strand containing any of natural or modified A, U, C, T, and G nucleotide.

A modified nucleotide can be, but is not limited to, d5SICS and dNaM that base pair with each other and dTPT3 also base pairs with dNaM (Floyd Romesberg), 2-amino-8-(2-thienyl)purine that base-pairs with pyridine-2-one (y), 7-(2-thienyl)imidazo[4,5-b]pyridine (Ds) that base-pairs with pyrrole-2-carbaldehyde (Pa), and Ds that base pairs with 4-[3-(6-aminohexanamido)-1-propynyl]-2-nitropyrrole (Px).

The oligonucleotide in the all-optical switches can be a single DNA or RNA strand, which include a donor, acceptor, and one or more photochromic nucleotides and can fold into such a conformation, so that the donor, acceptor, and photochromic nucleotide(s) are close enough to each other, so a photochromic Förster resonance energy transfer (pcFRET) can happen between the donor and acceptor and the photochromic nucleotide(s)' can affect such transfer.

As used herein, Förster resonance energy transfer (FRET), fluorescence resonance energy transfer (FRET), resonance energy transfer (RET), or electronic energy transfer (EET) refers to energy transfer between two light-sensitive molecules (donor and acceptor chromophores). A donor chromophore, initially in its electronic excited state, may transfer energy to an acceptor chromophore through nonradiative dipole-dipole coupling. The efficiency of this energy transfer is inversely proportional to the sixth power of the distance between donor and acceptor.

The all-optical switches disclosed herein can be a DNA or RNA duplex including two or more mostly matching or complementary oligonucleotides. In this situation, one of the two or more oligonucleotides can contain one of or all the photochromic nucleotide(s), donor, and acceptor, and the other can contain the rest. Since using a DNA or RNA duplex in the disclosed all-optical switches does not involving folding, a duplex is thereof preferred.

In some embodiments, two or more oligonucleotides forms one duplex. In some embodiments, two or more oligonucleotides forms two or more duplexes.

### Photochromic Nucleotide(s)

As used herein, a photochromic nucleotide refers to a nucleotide with a diarylethene, azobenzene, or its derivative attached to the base of the nucleotide. The diarylethene or azobenzene group can attached to the base of the nucleotide through a single bond to a nitrogen or carbon atom of the base.

A nucleoside consists simply of a nucleobase (also termed a nitrogenous base) and a five-carbon sugar (either ribose or deoxyribose), whereas a nucleotide is composed of a nucleobase, a five-carbon sugar, and one or more phosphate groups. In a nucleoside, the base is bound to either ribose or deoxyribose via a beta-glycosidic linkage.

Examples of nucleosides include cytidine, uridine, adenosine, guanosine, thymidine and inosine

For example, a

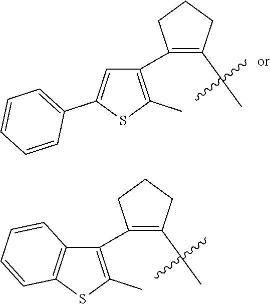 [SMILES: *C1=C(c2cc(-c3ccccc3)sc2C)CCC1 ; *C1=C(c2c(C)sc3ccccc23)CCC1]

group can be attached to a carbon atom of adenine, thymine, cytosine, or uracil, or to a nitrogen atom of guanine, adenine, thymine, cytosine, or uracil. For the photochromic nucleotides disclosed herein, the photoisomerization of the diarylethene or azobenzene group does not involve any proton from the base itself.

### Diarylethenes or Azobenzene

As used herein, a diarylethene or azobenzene group in the photochromic nucleotide can be any diarylethene or azobenzene group or its derivative that can undergo photoisomerization by different wavelengths.

Diarylethene is the general name of a class of compounds that have aromatic groups bonded to each end of a carbon-carbon double bond. The simplest example is stilbene, which has two geometric isomers, E and Z. A diarylethene group is a radical of diarylethene when a proton in the diarylethene is replaced by a bond.

Azobenzene is a chemical compound composed of two phenyl rings linked by a N═N double bond. An azobenzene group is a radical of diarylethene when a proton in the diarylethene is replaced by a bond.

As used herein, a diarylethene or azobenzene group includes the groups that are derived from substituted diarylethene or azobenzene.

### Donor or Acceptor

As used herein, a donor can be a fluorophore (or fluorochrome, similarly to a chromophore). A fluorophore absorbs light energy of a specific wavelength and re-emits light at a longer wavelength.

The absorbed wavelength, energy transfer efficiency, and time before emission of a specific fluorophore depend on both the fluorophore structure and its chemical environment, as the fluorophore in its excited state interacts with surrounding molecules. Wavelengths of maximum absorption (≈excitation) and emission (for example, Absorption/Emission=485 nm/517 nm) are the typical terms used to refer to a given fluorophore, but the whole spectrum may also be important for consideration. The excitation wavelength spectrum may be a very narrow or broader band, or it may be all beyond a cutoff level. The emission spectrum is usually sharper than the excitation spectrum, has a longer wavelength, and has correspondingly lower energy. Excitation energy of a fluorophore can range from ultraviolet through the visible spectrum, and emission energy can continue from visible light into the near infrared region.

Fluorophores typically contain several combined aromatic groups, or planar or cyclic molecules with several π bonds. A fluorophore that can be used in the all-optical switches disclosed herein is typically an organic small molecule of 20-100 atoms and has a molecular weight of from about 200 Da to about 1000 Da. In some embodiments, the fluorophore used as a donor or acceptor can have a molecular weight of from about 100 Da to about 2,000 Da, from about 300 Da to about 800 Da, from about 400 Da to about 600 Da, about 350 Da, about 400 Da, about 450 Da, about 500 Da, about 550 Da, or any value there between.

A fluorophore that can be used in the all-optical switches disclosed herein as a donor or acceptor include, but is not limited to, a xanthene derivatives such as fluorescein, rhodamine, oregon green, eosin, and Texas red; cyanine derivatives such as cyanine, indocarbocyanine, oxacarbocyanine, thiacarbocyanine, and merocyanine; a squaraine derivative or ring-substituted squaraines such as Seta, SeTau, and Square dyes; a naphthalene derivative such as a dansyl or prodan derivative; a coumarin derivative; a oxadiazole derivative such as pyridyloxazole, nitrobenzoxadiazole and benzoxadiazole; an anthracene derivatives such as anthraquinones including DRAQ5, DRAQ7 and CyTRAK Orange; a pyrene derivative such as cascade blue; an oxazine derivative such as Nile red, Nile blue, cresyl violet, oxazine 170; an acridine derivative such as proflavin, acridine orange, acridine yellow; and an arylmethine derivative such as auramine, crystal violet, and malachite green; a tetrapyrrole derivative such as porphin, phthalocyanine, and bilirubin.

A fluorophore that can be used in the all-optical switches disclosed herein as a donor or acceptor include, but is not limited to, a trademarked dye, such as a CF dye (Biotium); DRAQ or CyTRAK probes (BioStatus); BODIPY (Invitrogen); Alexa Fluor (Invitrogen); DyLight Fluor (Thermo Scientific, Pierce); Atto and Tracy (Sigma Aldrich); FluoProbes (Interchim); Abberior Dyes (Abberior); DY and MegaStokes Dyes (Dyomics); Sulfo Cy dyes (Cyandye); HiLyte Fluor (AnaSpec); Seta, SeTau and Square Dyes (SETA BioMedicals); Quasar and Cal Fluor dyes (Biosearch Technologies); SureLight Dyes (APC, RPEPerCP, PhycobilisomesxColumbia Biosciences); APC, APCXL, RPE, BPE (Phyco-Biotech, Greensea, Prozyme, Flogen); or Vio Dyes (Miltenyi Biotec).

The fluorophore can be covalently attached to a nucleotide of the oligonucleotide(s) or can be intercalated within the oligonucleotides.

As used herein, a donor in the all-optical switches disclosed herein is a fluorophore whose emission band overlaps to some degree with the absorbance band of the photochromic moiety. As used herein, an acceptor is a fluorophore whose absorbance band overlaps to some degree with the emission band of the donor. A specific example of an acceptor is the Alexa 488 dye used to demonstrate the all-optical excitonic switch.

In some embodiments, a donor that can be used in the all-optical switches disclosed herein include, but is not limited to, a dye, such as ATTO 390 dye, Eterneion 384/480 dye, DEAC (D-AMCA) dye, or a derivative thereof, which can be obtain from companies including IDT and Biosynthesis.

In some embodiments, an acceptor is an Alexa or ATTO 488 dye such as 6-FAM dye and Fluoresein dT dye, which can be obtain from companies including IDT and Biosynthesis.

The donor and acceptor can be chosen once the photochromic moiety of the specific all-optical switch and its corresponding wavelength are established.

### All-Optical Switches

As used herein, ON/OFF switching contrast refers the difference between the ON state static emission and the OFF state static emission of either the donor or the acceptor.

An all-optical switch disclosed herein ultimately output a modulated light signal, more specifically a modulation of the acceptor emission. Cyclic fatigue indicates any undesirable changes in the donor or the acceptor emission as a result of repeated ON/OFF cycling. Cycle time describes the time of exposure to a given wavelength of light (for example, 300 nm or 455 nm) used to cycle the all-optical excitonic switch between the closed or open configurations, respectively. Modulation refers to a statistically significant variation in donor or acceptor emission between the ON and OFF states. Photostability refers to the limited irreversible degradation of chromophores.

In order to observe modulation, in some embodiments, the donor and acceptor are placed within the Forster radius (they can be somewhat more or less). In some embodiments, the photochromic moiety should be placed between the acceptor and donor. However, in some embodiments, the photochromic moiety is positioned such that the donor can be between the photochromic moiety and acceptor, because it is possible that the photochromic moiety modulates both the donor and acceptor.

In some embodiments, to output, adjust, or modify a modulation light signal, an all-optical switch can have more than one photochromic nucleotide or photochromic moiety, different distance among the photochromic nucleotide (moiety), donor, and acceptor, and positions thereof, in addition to choose different photochromic nucleotide, donor, acceptor, and oligonucleotide(s).

An all-optical switch disclosed herein of course can output a modulation at various wavelengths by choosing the desired modulated emission wavelength first, then the appropriate photochromic moiety, donor, acceptor, and oligonucleotide(s).

Cyclic fatigue refers to any undesirable changes in the donor or the acceptor emission as a result of repeated ON/OFF cycling. Cycle time refers to the time of exposure to a given wavelength of light (i.e, 300 nm or 455 nm) used to cycle the all-optical excitonic switch disclosed herein between the closed or open configurations, respectively. An observable cyclic fatigue is about 1%, about 2%, about 3%, about 4%, about 5%, about 10%, about 15%, or more decreased or increased modulation. Photostability refers to the limited irreversible degradation of chromophores.

In some embodiments, an all-optical switch disclosed here does not exhibit observable cyclic fatigue after about 100 cycles, about 200 cycles, about 300 cycles, about 400 cycles, about 500 cycles, about 1,000 cycles, or any values there before.

An all-optical excitonic switch disclosed herein can be used as an optical analog to a metal-oxide-semiconductor field-effect transistor (MOSFET) or bipolar transistor. A MOSFET has a source, drain, and gate. The gate is electrically modulated to modulate the output of the drain whereby a voltage is applied to the drain, the source is at ground, and the gate is modulated by a voltage that is off then on then off.

Similarly, the donor of an all-optical excitonic switch can be thought of the source in MOSFET, the acceptor can be thought of as the drain, and the photochromic moieties can be thought of as the gate, respectively. As such, when the donor is under constant light illumination, the photochromic moieties can be modulated by modulating the illumination of its light source thereby causing the acceptor, thought of as the drain, to have a modulated emission.

A bipolar transistor has an emitter, base and collector. The donor of an all-optical excitonic switch can be thought of as the emitter, the photochromic moieties as the base, and the acceptor as the collector of a bipolar transistor, respectively. An all-optical excitonic switch through light can then be operated as a bipolar transistor.

An all-optical excitonic switch disclosed herein can be used as an optical nonvolatile memory device (i.e., flash memory). Because the diarylethene photochromic moieties are thermodynamically stable in either the “Open” or “Closed” configuration, either the “Open” or “Closed” configuration can be thought of as a permanent or stable “ON” or “OFF” memory state—that is, as a “0” or a “1”—that is nonvolatile. Hence, the all-optical excitonic switch can be an optical nonvolatile memory device analogous to the electronic flash memory device that incorporates floating gate memory.

To “Read” the optical excitonic nonvolatile memory device, the donor can be illuminated, and the output of the acceptor can then be examined to determine whether the state is either OFF or ON. To “Write” to the memory device or to “Erase” the memory device, the photochromic moiety is illuminated with light to cause it to either “Open” or “Close.”

In one aspect, disclosed herein is an all-optical excitonic switch, the switch comprises one, two or more oligonucleotides, wherein at least one oligonucleotide comprises one or more photochromic nucleotides; at least one oligonucleotide comprises a donor, and at least one oligonucleotide comprises an acceptor, wherein the photochromic nucleotide can undergo reversible photoisomerization under different wavelengths; an excitonic transfer occurs between emission of the donor and absorption of the acceptor; and the emission of the donor and the absorption of the acceptor fall within absorption of the one or more photochromic nucleotides.

In some embodiments, the switch comprises a single oligonucleotide. In some other embodiments, the switch comprises two oligonucleotides. In some other embodiments, the switch comprises three or more oligonucleotides. In yet some embodiments, the switch comprises two oligonucleotides that forms an oligonucleotide duplex. In yet some embodiments, the switch comprises three or more oligonucleotides that forms an oligonucleotide duplex. In yet some embodiments, the switch comprises two or more oligonucleotides that can be complementary to one another.

In some embodiments, the switch comprises a single oligonucleotide that can fold into a conformation.

In some embodiments, the oligonucleotide in the all-optical switches disclosed herein is a DNA, RNA, PNA, LNA, BNA polymer, or combination thereof. In some other embodiments, the oligonucleotide in the all-optical switches disclosed herein is a DNA polymer. In some embodiments, the oligonucleotide in the all-optical switches disclosed herein is a RNA polymer. In some embodiments, the oligonucleotide in the all-optical switches disclosed herein comprises one or more modified nucleotides.

In some embodiments, an all-optical switch disclosed herein comprises two oligonucleotides that form a DNA, RNA duplex, or mixture thereof. In some other embodiments, an all-optical switch disclosed herein comprises two oligonucleotides that form an RNA duplex. In yet some other embodiments, an all-optical switch disclosed herein comprises two oligonucleotides that form a DNA duplex. In some embodiments, an all-optical switch disclosed herein comprises three or more oligonucleotides that form one or more DNA, RNA duplex, or mixture thereof. In some other embodiments, an all-optical switch disclosed herein comprises three or more oligonucleotides that form one or more RNA duplex. In yet some other embodiments, an all-optical switch disclosed herein comprises three or more oligonucleotides that form one or more DNA duplex.

In some embodiments, the photochromic nucleotide in an all-optical switch disclosed herein is a modified nucleotide having a modified nitrogenous base. In some other embodiments, the photochromic nucleotide in an all-optical switch disclosed herein comprises a modified adenine, guanine, cytosine, thymine, or uracil base. In yet some other embodiments, the photochromic nucleotide in an all-optical switch disclosed herein comprises a modified uracil or cytosine base.

In some embodiments, the photochromic nucleotide in an all-optical switch disclosed herein comprises a diarylethene group. In some other embodiments, the photochromic nucleotide in an all-optical switch disclosed herein comprises an azobenzene group.

In some embodiments, the photochromic nucleotide in an all-optical switch disclosed herein comprises a base, wherein the base comprises a

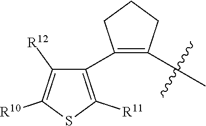

group, wherein R¹⁰ and R¹² are independently H, CH₃, an alkyl, or together a ring; and wherein R¹¹ is H, CH₃, or an alkyl.

In some embodiments, the photochromic nucleotide comprises a base of

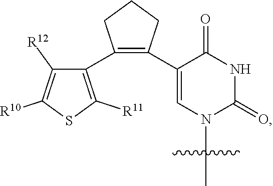

wherein R¹⁰ and R¹² are independently H, CH₃, an alkyl, or together a ring; and wherein R¹¹ is H, CH₃, or an alkyl.

In some embodiments, the photochromic nucleotide in an all-optical switch disclosed herein comprises a

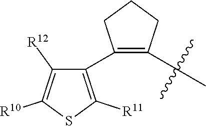

group or a base

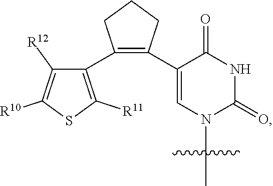

wherein R¹⁰ is aryl group and R¹¹ and R¹² are independently H, CH₃, or alkyl. In some other embodiments, R¹⁰ and R¹² are independently H, CH₃, an alkyl, or together a ring; and wherein R¹¹ is H, CH₃, or an alkyl. In other embodiments, R¹⁰ and R¹² are together a C₅ or C₆ member ring and R¹¹ is H, CH₃, or alkyl. In some other embodiments, R¹⁰ and R¹² are together a substituted or unsubstituted aromatic ring and R¹¹ is H, CH₃, or alkyl. In some embodiments, R¹⁰ is aryl group; R¹¹ is CH₃; and R¹² is H. In yet some other embodiments, R¹⁰ is —C₆H₅; R¹¹ is CH₃; and R¹² is H. In some embodiments, R¹⁰ is a substituted aryl group; R¹¹ is CH₃; and R¹² is H.

In some embodiments, the all-optical switch disclosed herein comprises two photochromic nucleotides. In some other embodiments, the all-optical switch disclosed herein comprises three photochromic nucleotides. In yet some other embodiments, the all-optical switch disclosed herein comprises four or more than four photochromic nucleotides.

In some embodiments, the two or more photochromic nucleotides are in the same oligonucleotide. In some other embodiments, the two or more photochromic nucleotides are in different oligonucleotides.

In some embodiments, the one or more photochromic nucleotides are

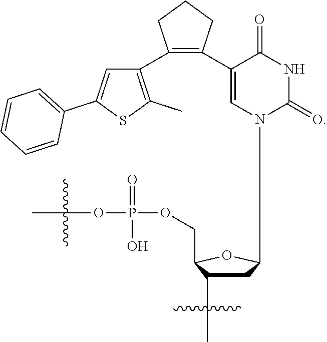

In some embodiments, the oligonucleotide comprises a CXA CXA or CXA CXA CXA sequence, wherein X is a photochromic nucleotide. In some other embodiments, the oligonucleotide comprises a CdU^{ps} A CdU^{ps} A or CdU^{ps} A CdU^{ps} A CdU^{ps} A sequence, wherein dU^{ps} is

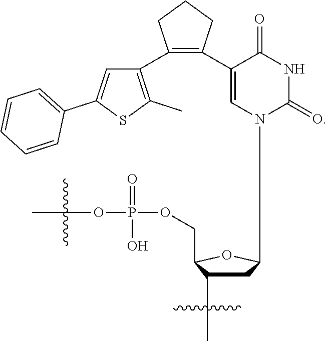

In some other embodiments, wherein the oligonucleotide comprises a GGC TAG CGA CdU^{ps} ACdU^{ps} A (SEQ ID NO: 6) or GGC TAG CdU^{ps} A CdU^{ps} A CdU^{ps} A (SEQ ID NO: 4) sequence, wherein dU^{ps} is

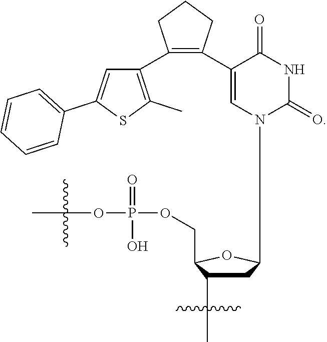

In some embodiments, the donor or acceptor of the all-optical switch disclosed herein is a fluorophore. In some other embodiments, the donor is an ATTO dye. In some other embodiments, the donor is A390 dye.

In some embodiments, the acceptor is an ATTO dye. In some other embodiments, the acceptor is A488 dye. In some other embodiments, the donor is A390, the acceptor is A488, and the photochromic nucleotide is dU^{ps}.

In some embodiments, the oligonucleotide comprises a donor-AGT AGT AGC TAG CCG CAC GCA CCG GCT CG sequence (SEQ ID NO: 1).

In some embodiments, the oligonucleotide comprises a CGA GCC GGT GCG TGC-acceptor sequence (SEQ ID NO: 2).

In some embodiments, the donor of the all-optical switches disclosed herein has an emission band of from about 400 nm to about 550 nm.

In some embodiments, the acceptor has absorption band of from about 450 nm to about 550 nm.

In some embodiments, the photochromic nucleotide(s) has absorption band of from about 370 nm to about 570 nm.

In some embodiments, the all-optical switch disclosed herein can operate in solid state. In some other embodiments, the all-optical switch disclosed herein can operate in liquid state.

The all-optical switch disclosed herein outputs a modulation of a light signal. As used herein, “modulation” refers to changes of the acceptor's emission in amplitude, wavelength, or both, when the all-optical switch disclosed herein is switch from “ON” state to “OFF” state or vis-versa.

In some embodiments, the light signal has a wavelength from ultra violet to far red light, from about 100 nm to about 10,000 nm, from about 200 nm to about 2,000 nm, from about 400 nm to about 1,000 nm, from about 400 nm to about 800 nm, from about 400 to about 700 nm, from about 500 nm to about 600 nm, or any value there between.

In another aspect, disclosed herein is a method for creating an “ON” or “Off” state, wherein the method comprising shining a first light and then second light to the all-optical switch disclosed herein, wherein the all-optical switch emits an emission light signal and the emission light signal varies depending on if the first light and second light are on at the same time.

In yet another aspect, disclosed herein is a device for data processing or storing information, wherein the device comprises one or more all-optical switches disclosed herein and the all-optical switches function as flash memory or transistor.

As used herein, the term “substantially free”, “free” or “free of” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-% and in yet another embodiment, the amount of component is less than 0.01 wt-%.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The methods and compositions of the present disclosure may comprise, consist essentially of, or consist of the components and ingredients of the disclosed compositions or methods as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

### EXAMPLES

Embodiments of the present disclosure are further defined in the following non-limiting Examples. These Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Chemicals and Analytical Techniques

Commercially available reagents were purchased from Sigma Aldrich, Carbolution, and Carbosynth. These reagents and laboratory grade solvents were used without further purification. Dry (moisture-free) solvents were purchased in sealed bottles packaged over molecular sieves. Solvents for oxygen-sensitive reactions were degassed prior to use by passing a continuous argon flow through the solvent for about 10 minutes. All reactions were monitored by thin layer chromatography (TLC) using silica plates coated with a fluorescent indicator and visualized with UV light (254 nm) or stained with a blue shift solution (ceric sulfate, molybdatophosphoric acid, and sulfuric acid). Reactions under high pressure were conducted in microwave tubes sealed with a gas tight climber cap. Flash chromatography was performed with silica gel 60 (0.04-0.063 mm) using laboratory grade solvents. High-resolution mass spectra were recorded on a Bruker microTOF-Q II ESI (ESI). Nuclear magnetic resonance (NMR) spectra were recorded on Varian Systems 300 and 500 instruments with chemical shifts (8) indicated in parts per million (ppm) downfield of tetramethylsilane (TMS) and referenced to the respective residual un-deuterated solvent peak as follows: CDCl₃=7.26 ppm, MeOH-d₄=3.31 ppm for ¹H-NMR; and CDCl₃=77.0 ppm, MeOH-d₄=49.00 ppm for ¹³C-NMR. Apparent coupling constants (J) are reported in Hz.

### Example 1

### Synthesis and Characterization of the Exemplary Phosphoramidite for Solid Phase Synthesis

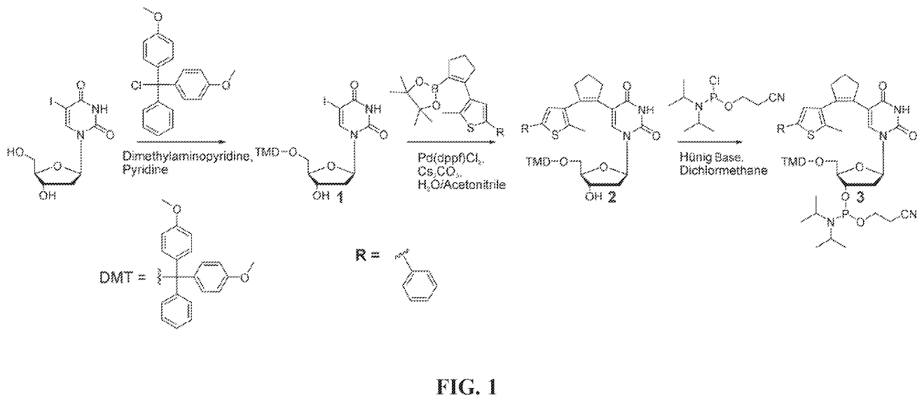

A general scheme for the synthesis of an exemplary phosphoramidite for solid phase synthesis is shown in FIG. 1. The specific procedure and analytical data for the exemplary product and its precursors are listed below.

### 1-((2R,4S,5R)-5-((bis(4-methoxyphenyl)(phenyl)methoxy)methyl)-4-hydroxytetrahydrofuran-2-yl)-5-iodopyrimidine-2,4(1H,3H)-dione (1)

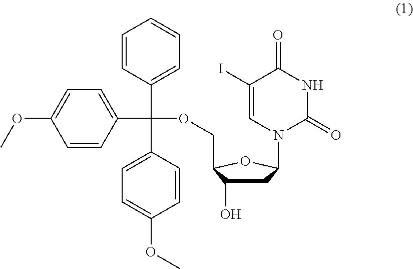

In a Schlenk flask under argon 5-iodo-2′-deoxy-uridine (5 g, 14.12 mmol) was dissolved in dry pyridine (80 mL). 4,4-Dimethoxytritylchloride (DMT-Cl) (5.76 g, 17.01 mmol) was added and the mixture stirred overnight at room temperature. Ice water was added, and the reaction mixture extracted with dichloromethane (DCM). The combined organic phases were washed with water and brine, dried over MgSO4, filtered, and the solvent removed under reduced pressure. Purification by flash column chromatography (silica gel, DCM/MeOH 50:1+1% NEt₃) afforded (1) as a white solid with a yield of 85% (7.9 g, 12.0 mmol).

¹H NMR (300 MHz, methanol-d4): δ=8.20 (s, 1H), 7.51-7.18 (m, 9H), 6.93-6.83 (m, 4H), 6.23 (dd, J=7.5, 6.0 Hz, 1H), 4.48 (dt, J=5.8, 2.9 Hz, 1H), 4.05 (q, J=3.2 Hz, 1H), 3.79 (s, 6H), 3.36 (d, J=3.3 Hz, 2H), 2.46-2.27 (m, 2H).

### 1-((2R,4S,5R)-5-((bis(4-methoxyphenyl)(phenyl)methoxy)methyl)-4-hydroxytetrahydrofuran-2-yl)-5-(2-(2-methyl-5-phenylthiophen-3-yl)cyclopent-1-en-1-yl)pyrimidine-2,4(1H,3H)-dion (2)

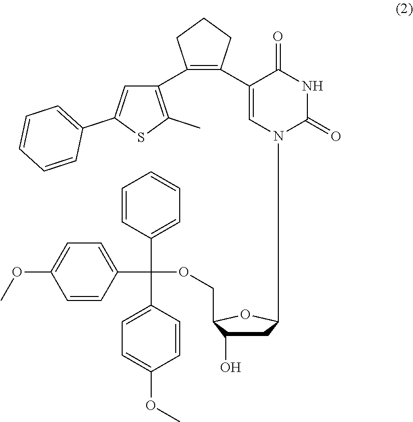

In a microwave vial compound (1) (100 mg, 0.15 mmol), 4,4,5,5-tetramethyl-2-(2-(2-methyl-5-phenylthiophen-3-yl)cyclopent-1-enyl)-1,3,2-dioxaborolane, (110 mg, 0.31 mmol), Pd(dppf)Cl₂ (7 mg, 8 μmol), and Cs₂CO₃ (250 mg, 0.8 mmol) were dissolved in acetonitrile (2 mL) and water (1 mL) under argon. The mixture was degassed 3 times, the vial sealed, and stirred at 120° C. for 1 hour. The reaction mixture was absorbed on silica and purified by flash column chromatography (silica gel, DCM/MeOH 20:1+1% NEt₃), affording (2) as yellowish solid in a yield of 90% (104 mg, 0.13 mmol).

¹H NMR (300 MHz, methanol-d4): δ=7.60-7.18 (m, 14H), 7.11 (s, 1H), 6.88-6.77 (m, 4H), 6.70 (s, 1H), 6.18 (t, J=6.6 Hz, 1H), 3.86 (dt, J=7.4, 3.7 Hz, 1H), 3.80 (s, 3H), 3.75 (s, 3H), 3.63 (dt, J=7.7, 4.0 Hz, 1H), 3.04 (ddd, J=24.9, 10.2, 3.1 Hz, 2H), 2.84 (dt, J=14.0, 7.2 Hz, 1H), 2.60 (dt, J=14.8, 7.4 Hz, 2H), 2.16-2.08 (m, 1H), 2.07 (s, 3H), 2.05-2.00 (m, 1H), 1.57 (dt, J=13.9, 7.1 Hz, 2H). ¹³C NMR (125 MHz, methanol-d₄): δ=164.60, 160.15, 151.50. 146.24, 142.03, 138.94, 137.96, 137.30, 137.24, 136.74, 135.32, 134.58, 134.48, 133.37, 131.41, 131.21, 130.07, 129.34, 128.87, 128.43, 127.82, 126.28, 125.20, 114.21, 114.13, 113.14, 87.48, 87.16, 85.56, 75.83, 72.11, 65.70, 55.77, 41.49, 38.86, 25.03, 23.64, 14.12. HRMS (ESI, positive) m/z: [M+Na]⁺ calc. for [C₄₆H₄₄N₂O₇SNa]⁺: 791.2761, found: 791.2725.

### (2R,3R,5R)-2-((bis(4-methoxyphenyl)(phenyl)methoxy)methyl)-5-(5-(2-(2,5-dimethylthiophen-3-yl)cyclopent-1-en-1-yl)-2,4-dioxo-3,4-dihydropyrimidin-1(2H)-yl)tetrahydrofuran-3-yl(2-cyanoethyl) diisopropylphosphoramidite (3)

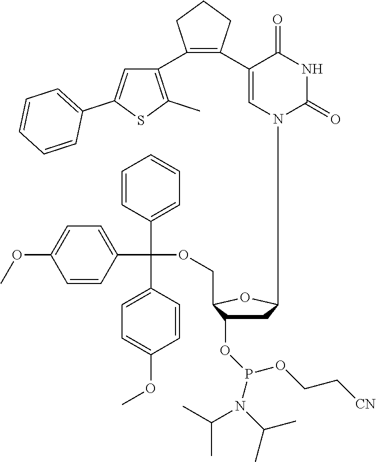

In a Schlenk flask under argon compound (2) (520 mg, 0.68 mmol) and Hünig's base (0.18 mL, 1 mmol) were dissolved in dry DCM (10 mL) and cooled down to 0° C. 2-cyanoethyl N,N-diisopropylchlorophosphoramidite (CEP-Cl, 0.31 mL, 1.1 mmol) was added, and the mixture was stirred at room temperature for 3 hrs. Purification via flash column chromatography (silica gel, DCM/MeOH 100:1+1% NEt₃) afforded (3) as a white solid with a yield of 84% (550 mg, 0.57 mmol) as a mixture of isomers.

¹H NMR (500 MHz, CDCl₃): δ=7.42-7.36 (m, 2H), 7.26 (s, 11H), 7.01 (d, J=1.2 Hz, 1H), 6.79 (ddd, J=8.8, 6.8, 2.0 Hz, 4H), 6.69 (d, J=3.3 Hz, 1H), 6.18 (ddd, J=7.8, 5.7, 1.9 Hz, 1H), 4.25-4.08 (m, 1H), 4.07-4.01 (m, 1H), 3.97 (tt, J=6.8, 3.9 Hz, 1H), 3.95-3.84 (m, 2H), 3.79 (dd, J=6.0, 4.7 Hz, 5H), 3.68-3.39 (m, 6H), 3.11 (dtd, J=10.6, 6.5, 3.1 Hz, 2H), 3.01 (tt, J=11.1, 5.6 Hz, 2H), 2.83-2.70 (m, 2H), 2.71-2.53 (m, 4H), 2.46 (t, J=6.5 Hz, 1H), 2.30-2.18 (m, 2H), 2.08-2.01 (m, 4H), 1.50-1.41 (m, 1H), 1.38 (d, J=13.8 Hz, 3H), 1.33 (t, J=7.4 Hz, 1H), 1.29-1.21 (m, 8H), 1.20-1.14 (m, 4H), 1.11 (dd, J=11.6, 6.8 Hz, 5H), 1.04 (d, J=6.8 Hz, 3H), 0.92 (d, J=6.8 Hz, 3H). ¹³C NMR (125 MHz, CDCl₃): δ=161.64, 158.76, 158.65, 149.50, 149.49, 144.61, 144.61, 140.73, 140.69, 137.49, 137.43, 137.11, 137.09, 136.23, 136.22, 135.50, 135.49, 134.20, 134.16, 133.63, 133.51, 133.00, 132.97, 130.45, 130.38, 130.08, 130.03, 129.08, 128.40, 128.34, 128.05, 128.04, 127.38, 127.36, 127.03, 126.99, 125.45, 125.39, 124.20, 124.14, 117.54, 117.46, 117.01, 113.31, 113.29, 112.43, 112.42, 110.16, 86.47, 86.46, 85.24, 85.21, 85.03, 84.98, 84.23, 84.20, 77.16, 73.78, 73.65, 73.46, 73.32, 64.11, 64.02, 64.01, 58.42, 58.40, 58.31, 58.27, 58.25, 55.42, 55.40, 55.37, 45.50, 45.45, 43.44, 43.36, 43.34, 43.26, 39.34, 38.13, 36.05, 36.03, 25.39, 25.37, 25.04, 25.03, 24.97, 24.72, 24.69, 24.66, 24.64, 24.59, 24.54, 24.53, 24.48, 23.15, 23.13, 23.06, 23.04, 22.80, 22.78, 20.43, 20.37, 20.30, 20.24, 20.17, 20.12, 14.17, 14.15. ³¹P NMR (202 MHz, CDCl₃): δ=149.34, 148.94. HRMS (ESI, positive) m/z: [M+Na]⁺ calc. for [C₅₅H₆₁N₄O₈SPNa]⁺: 991.3840, found: 991.3814.

### Example 2

### Solid Phase Synthesis of Exemplary Modified Oligonucleotides

The oligonucleotides comprising modified nucleoside(s), specifically, photochromic strands (PS1 and PS3), were synthesized by solid phase synthesis using phosphoramidite chemistry on an Expedite 8909 synthesizer. Table 1 lists the exemplary sequences and some characteristics of the exemplary oligonucleotides containing one or three modified nucleosides, respectively.

TABLE 1
Mass, sequence and chemical formula of the synthesized oligonucleotides.

| | | | calculated | found |
|---|---|---|---|---|
| Name | Sequence | chemical formula | mass [m/z] | mass [m/z] |
| PS1 | GGC TAG CTA | C₁₆₁H₁₉₆N₅₈O₈₇P₁₄S₁ | 4800.8807 | 4800.8795 |
| | CdU^{PS}A CGA | | | |
| | (SEQ ID NO: 3) | | | |
| PS3 | GGC TAG | C₁₉₁H₂₂₁N₅₅O₈₈P₁₄S₃ | 5224.0049 | 5223.9483 |
| | CdU^{PS}A CdU^{PS}A | | | |
| | CdU^{PS}A (SEQ ID | | | |
| | NO: 4) | | | |

In the above Table 1, dU^{PS} is the acronym for

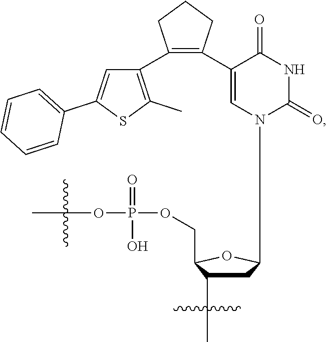

deoxyuridine-based photoswitchable nucleoside with phenyl substituent.

Other reagents were purchased from Roth and Sigma Aldrich (Proligo) and used without further purification. A solid support 500 Å controlled pore glass (CPG) was used. The phosphoramidites of the four unmodified DNA-nucleosides (2′-deoxy-adenosine, -guanosine, -cytidine and -thymine) were dissolved in dry acetonitrile at a concentration of 0.075 M and molecular sieves (Roth, 4 Å, type 514) were added. The modified phosphoramidite (3) from Example 1 was dissolved in dry acetonitrile at a concentration of 0.1 M. For the synthesis of the modified oligonucleotides, a standard 1 μmol-protocol was used. After the synthesis, the oligonucleotide was deprotected and cleaved off the CPG using 25% ammonia (4 hours, 40° C.). The solid phase was washed with 25% ammonia (3×1 mL) and ultrapure water (3×1 mL) to remove the oligonucleotide completely from the solid support. The ammonia/water mixture was lyophilized, and the crude product was dissolved in water, filtered, and purified using preparative HPLC.

### HPLC Purification

HPLC purification and characterization were performed using an Agilent HP 1100 series machine equipped with a diode array detector (DAD) and processed using the ChemStation software. Purification was accomplished using a semi-preparative Luna 5u C18(2) 100 A column (15×250 mm, Phenomenex) with a flowrate of 5 mL/min. The mobile phases were Buffer A (0.1 M triethylammonium acetate in water, pH 7) and Buffer B (0.1 M triethylammonium acetate in 80% acetonitrile). The HPLC methods (A and B; for PS1 and PS3, respectively) are listed below in Table 2.

TABLE 2
Gradients for preparative HPLC purification of the
synthesized oligonucleotides

| Method A | | Method B | |
|---|---|---|---|
| Time [min] | Buffer B [%] | Time [min] | Buffer B [%] |
| 0 | 10 | 0 | 5 |
| 5 | 20 | 2 | 5 |
| 10 | 30 | 7 | 15 |
| 35 | 40 | 22 | 20 |
| 40 | 100 | 29 | 40 |
| 42 | 10 | 35 | 60 |
| | | 45 | 100 |
| | | 55 | 5 |

The product-containing fractions were lyophilized and their purity was analyzed by HPLC using Method C listed in Table 3. In Method C, the mobile phases employed were Buffer C (100 mM ‘hexafluoroisopropanol+8.6 mM triethylamine, pH 8.3) and methanol (LC-MS grade, Sigma Aldrich).

TABLE 3
HPLC gradient for LC/MS measurements
Method C

| Time [min] | Methanol [%] |
|---|---|
| 0 | 10 |
| 5 | 20 |
| 10 | 30 |
| 35 | 40 |
| 40 | 100 |
| 42 | 10 |

### LC/MS Analysis.

Liquid chromatography/mass spectrum (LC/MS) characterization were carried out using an HP 1200 series HPLC from Agilent coupled to a microTOF-Q II ESI mass spectrometer (Bruker) and processed with the Hyphenation Star PP (Version 3.2.44.0) Software (Bruker Daltonics). A Kinetex 2.6u C18100 Å column (Phenomenex) was used.

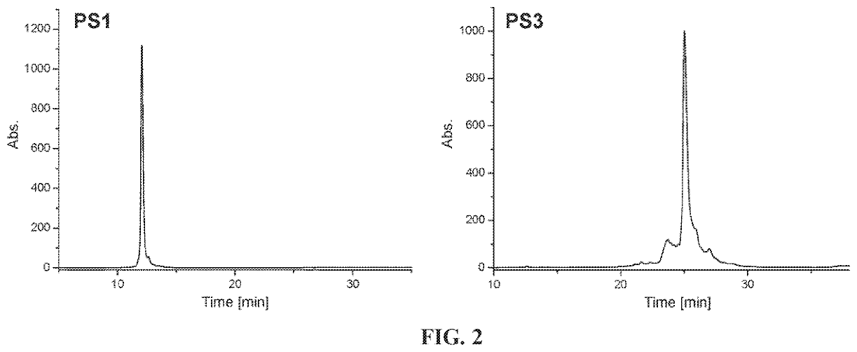

HPLC chromatograms of the synthesized modified oligonucleotides with one modification (PS1) and three modifications (PS3), detected at 280 nm are shown in FIG. 2.

### Example 3

Preparation of Exenplary Single Stranded DNA (ssDNA) Sequences with or without a Donor and/or Acceptor

The exemplary base sequences for an ATTO 390 donor and ALEXA 488 acceptor ssDNA strands in this Example were designed using the web-based NUPACK (http://nupack.org/) program, which allowed for matching its DNA bases to the custom photochromic nucleotide strand and/or each other (donor and acceptor strand). Furthermore, NUPACK provided thermodynamic analysis to determine the lowest free energy of the fully hybridized double stranded DNA structure. Some exemplary chromophore labeled (i.e., donor and acceptor), photochromic strands (nucleotide dU^{PS}), and control (i.e., bare) oligomers are listed in Table 4. In Table 4, ATTO 390 is (2,5-dioxopyrrolidin-1-yl) 4-(4,6,8,8-tetramethyl-2-oxo-6,7-dihydropyrano[3,2-g]quinolin-9-yl)butanoate and ALEXA 488 is Xanthylium, 3,6-diamino-9-[2-carboxy-4(or 5)-[[(2,5-dioco-1-pyrrolidinyl)oxy]carbonyl]phenyl]-4,5-disulfo-, inner salt, lithium salt (1:2).

TABLE 4
List of Exemplary Single strand DNA sequences

| | | Length | |
|---|---|---|---|
| Strand Name | Sequence (5′ to 3′) | (nt) | Purification |
| Photochromic | GGCTAGCTACdU^{PS}ACGA (SEQ ID NO: 3) | 15 | HPLC^{a} |
| Strand (1) - PS1 | | | |
| Photochromic | GGCTAGCdU^{PS}ACdU^{PS}ACdU^{PS}A (SEQ ID NO: 4) | 15 | HPLC^{a} |
| Strand (3) - PS3 | | | |
| Donor Strand | /A390/AGTAGTAGCTAGCCGCACGCACCGGCTCG | 29 | Dual |
| | (SEQ ID NO: 1) | | HPLC^{a} |
| Acceptor Strand | CGAGCCGGTGCGTGC/A488/ (SEQ ID NO: 2) | 15 | Dual |
| | | | HPLC^{a} |
| Photochromic | GGCTAGCTACTACTA (SEQ ID NO: 5) | 15 | Standard |
| (control) | | | Desalting^{b} |
| Donor (control) | AGTAGTAGCTAGCCGCACGCACCGGCTCG (SEQ | 29 | Standard |
| | ID NO: 1) | | Desalting^{b} |
| Acceptor (control) | CGAGCCGGTGCGTGC (SEQ ID NO: 2) | 15 | Standard |
| | | | Desalting^{b} |

^{a}High-Performance Liquid Chromatography
^{b}Desalting to remove short products and small organic contaminants. Does not include polyacrylamide gel electrophoresis (PAGE) purification.

Donor and acceptor chromophore strands were purchased as lyophilized powders from BioSynthesis (Lewisville, Tex., USA) with an initial synthesis scale at 1.0 optical density (OD) purified using Dual High-Performance Liquid Chromatography (HPLC). Control strands were purchased lyophilized from Integrated DNA Technologies (Coralville, Iowa, USA) with an initial synthesis scale at 250 nmole purified with a standard desalting process. All strands were re-hydrated using ultra-pure water (Barnstead Nanopure, Thermo Scientific) to a nominal 100 μM stock concentration and used without further purification.

### Example 4

Assembly of an Exemplary all-Optical Switch

Assembly of the exemplary all optical switch was performed with all ssDNA in 1× TAE buffer (40 mM tris(hydroxymethyl)aminomethane, 20 mM acetic acid, 2 mM ethylenediaminetetraacetic acid (EDTA); pH 8.0) with 15 mM magnesium acetate tetrahydrate (Mg²⁺) added at working DNA concentrations of 5 μM or 20 μM. The all-optical excitonic switch devices were constructed via self-assembly by combining all strands at equimolar concentration in a microcentrifuge tube and allowing ˜15 minutes of hybridization time at room temperature, then testing without further modification or purification. When the three strands hybridize, a linear excitonic transmission line approximately 4.76 nm in length is formed, placing all of the optical active components within their Förster distances.

### Example 5

Normalized and Unaltered Spectral Overlap Data and Chromophore Selection of an Exemplary all-Optical Switch

The chromophore (donor or acceptor) strands were selected such that excitonic transfer occurs between the donor emission and acceptor absorption. Additionally, the donor emission and the acceptor absorption fall well within the broad absorption band of the photochromic nucleotide.

Spectral overlap is required between all optical molecules involved in the FRET and excitonic transfer processes. Sufficient overlap between the emission spectrum of the donor and the absorption spectrum of the acceptor must be present. Furthermore, the absorption spectrum of the photochromic nucleotide must coincide with both the emission of the donor and the absorption of the acceptor.

Optical molecules were selected such that overlap occurred among the donor emission, photochromic nucleotide closed configuration absorption, and acceptor absorption in order to modulate both the excitonic transfer and FRET processes. Exposure wavelengths were chosen such that minimal perturbation to the configuration of the photochromic nucleotides would occur during data collection.

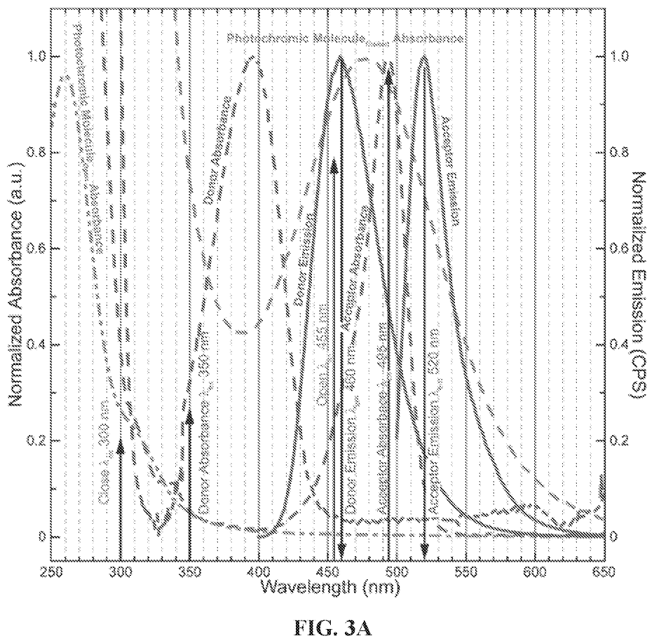

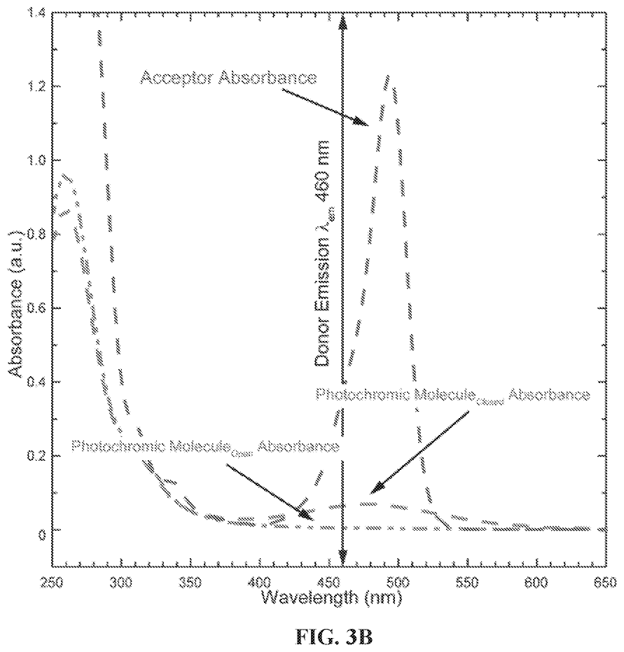

FIG. 3A shows normalized absorption and emission spectra of all chromophores present in the all-optical excitonic switch, the chromophores were selected such that facile excitonic transfer occurs between the ATTO 390 donor emission (λ max=460 nm, solid blue curve) and Alexa 488 acceptor absorption (λ max=495 nm, dashed green curve). FIG. 3A shows the normalized absorption and emission spectra of the donor strand (blue dashed and solid curves, respectively), normalized absorption spectra of the photochromic nucleotides in both the open (non-absorbing, red dashed/dotted curve) and closed (absorbing, red dashed curve) configurations, and normalized absorption and emission spectra of the acceptor strand (green dashed and solid curves, respectively). Up arrows indicate the wavelengths used to expose the optical molecule and the down arrow indicates the wavelength used to collect spectral data. The spectra have been normalized in order to easily visualize the spectral overlap in the 350-550 nm region.

Additionally, the donor emission and the acceptor absorption fall well within the broad absorption band of the photochromic nucleotide when it is in the closed (“OFF” state) configuration (FIG. 3A, red dashed curve, 388-550 nm). For additional clarity in understanding the relative absorbance of the acceptor and photochromic nucleotide moieties, which affects the overall switching efficiency (i.e., FRET modulation) of the all-optical excitonic switch,

FIG. 3B shows the unaltered (i.e., non-normalized) absorption spectra of the Alexa 488 acceptor strand as well as the three photochromic nucleotide switches in their open and closed configurations, with the peak emission wavelength of the ATTO 390 donor indicated. The vertical double-headed arrow indicates the donor's peak emission wavelength of 460 nm. Note that the absorbance of the photochromic nucleotides is ˜6× less than that of the acceptor at the donor's emission maximum, which leads to incomplete FRET quenching in the all-optical excitonic switch's OFF state (i.e., partial modulation of the fluorescence emission).

### Example 6

### Absorbance of One Versus Three Photochromic Nucleotides.

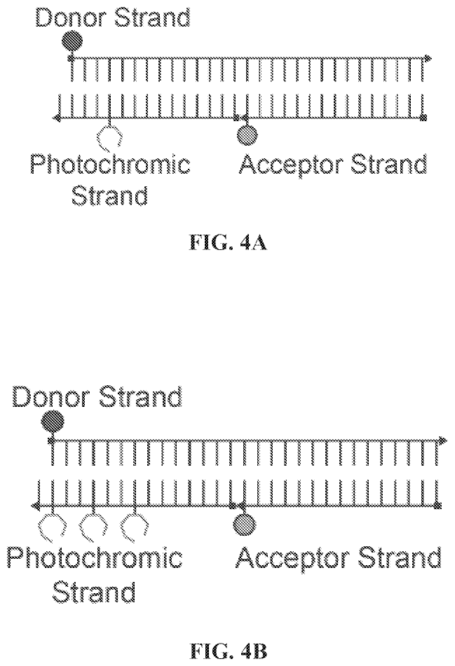

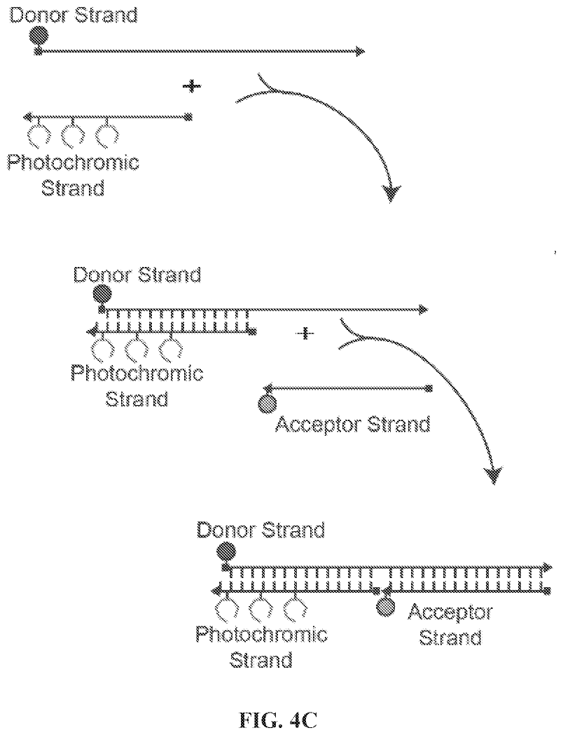

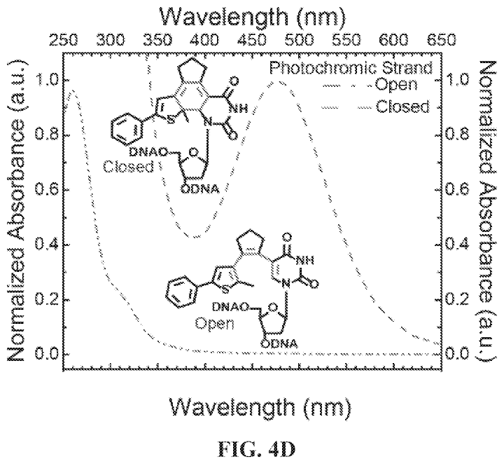

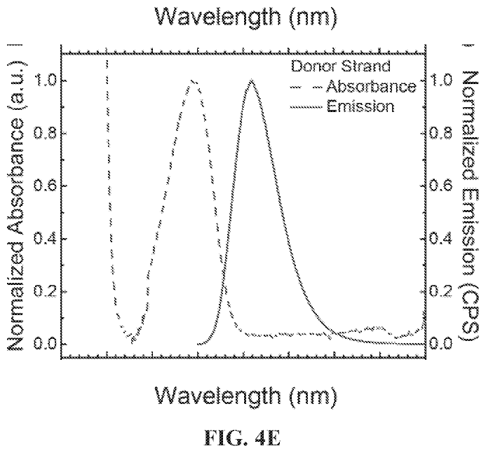

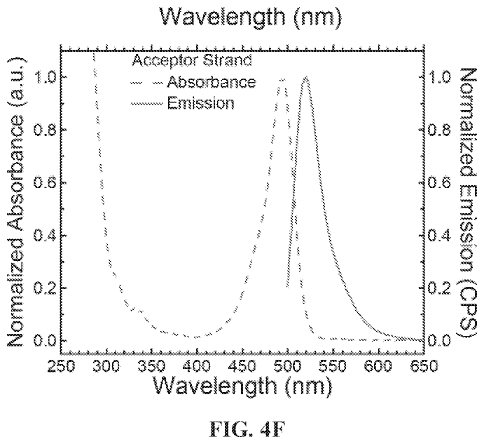

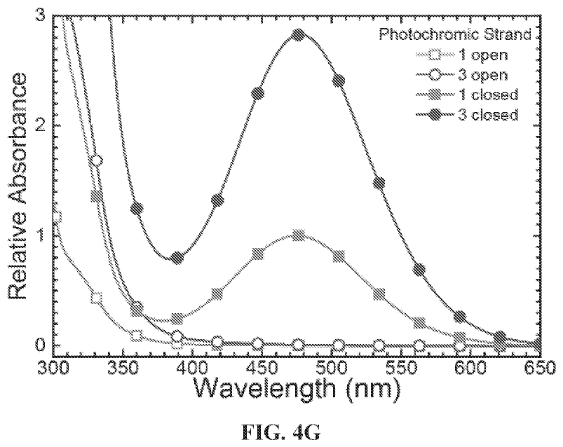

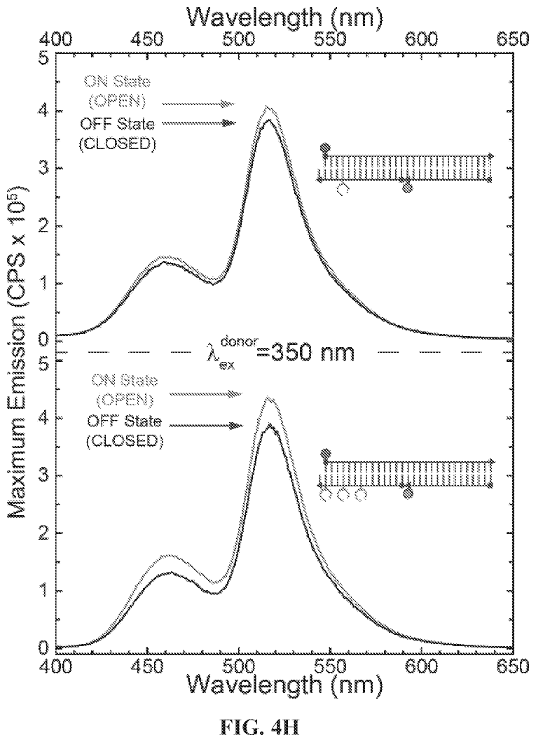

Photochromic strands PS1 and PS3 were hybridized with the donor and acceptor strands to generate two all-optical excitonic switches in different configurations as shown in FIG. 4A and FIG. 4B. The self-assembly of the all-optical excitonic switches is illustrated in FIG. 4C, wherein all positions are precisely controlled through sequence design, for example, using a procedure presented in Example 3.

In the photochromic nucleotides used in this Example, an uracil ring embedded in a DNA sequence is one of the aryl residues, attached via its 5-position to a cyclopentene-linked thiophene as shown in FIG. 4D. The premise behind the design of our all-optical excitonic switch is that the photochromic nucleotide(s) will modulate (i.e., sufficiently quench) the excitonic emission of both the donor and the acceptor when the donor, photochromic nucleotide(s), and acceptor are all positioned within the Förster radius of each other.

Based on the assumption that increasing the number of photochromic nucleotides present will increase the modulation of exciton flow and photon emission, the incorporation of one, two, three, or more photochromic nucleotides were possible. In this Example, exemplary all-optical switches comprising one and three photochromic nucleotides were examined (FIG. 4A and FIG. 4D).

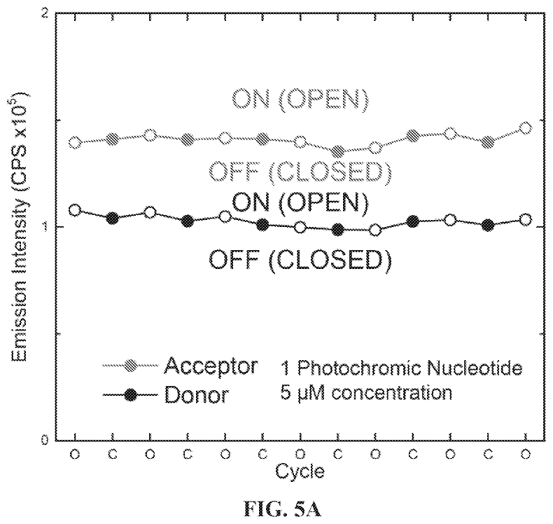

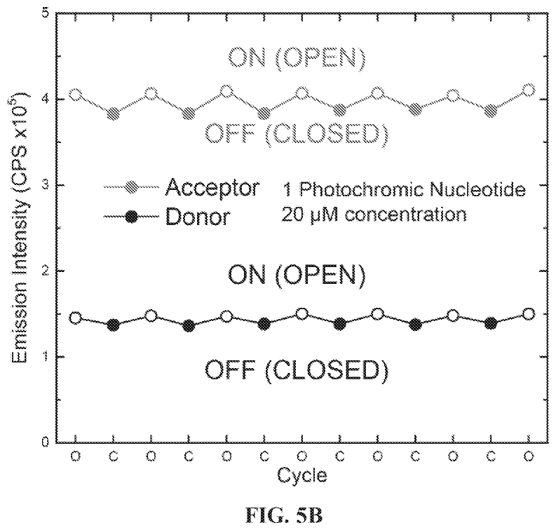

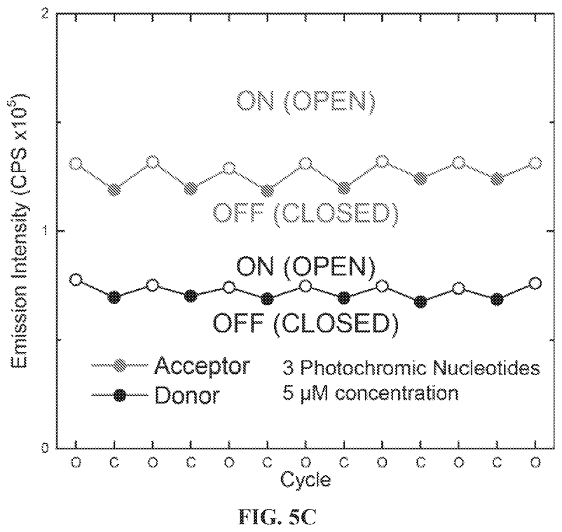

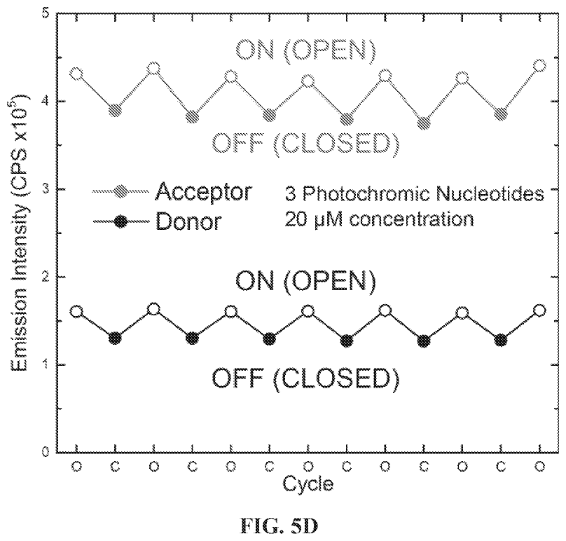

The expectation was that incorporation of three photochromic nucleotides would produce a three-fold increase in the donor-acceptor emission modulation. FIG. 5A-FIG. 5D shows the saw-tooth plots created from the emission data collected for 5 μM (FIG. 5A and FIG. 5B) and 20 μM (FIG. 5C and FIG. 5D) concentrations in the liquid environment with either one or three photochromic nucleotides attached.

FIG. 5A-FIG. 5D show saw-tooth plots of the all-optical excitonic switch operating in the liquid phase. FIG. 5A-FIG. 5D demonstrated the changes in the donor and acceptor emission as the photochromic nucleotides were cycled between the open and closed configuration (i.e., ON and OFF states) six times. FIG. 5A demonstrated the minimal modulation between the ON and OFF states of the 5 μM switch with a specific single photochromic nucleotide (strand PS1). FIG. 5B demonstrates 6.6% modulation between the ON and OFF state for a 20 μM switch with the PS1 strand. The weak modulation noted in FIG. 5A may be due to the emission of the donor/acceptor pair overwhelming the single photochromic nucleotide as well as the low absorbance of the specific single photochromic nucleotide. FIG. 5C demonstrated a 8.2% modulation between the ON and OFF states of a 5 μM switch with three specific photochromic nucleotides attached (PS3 strand). FIG. 5D demonstrates the greatest amount of donor/acceptor emission modulation (15.1%) of a 20 μM switch with the PS3 strand. The increased modulation resulted from the greater absorbance provided by the three photochromic nucleotides.

As shown in FIG. 5A-FIG. 5D, when the single photochromic nucleotide was used, the donor and acceptor emission was compared after each opening and closing event to reveal any saw-tooth behavior indicative of modulation. For the 5 μM single photochromic nucleotide sample, modulation is initially barely visible (1.0% and 0.6%) but disappears during later cycling. However, for the 20 μM sample, a 6.6% and 5.0% saw-tooth modulation was observed. When three photochromic nucleotides were present, clear modulation of the donor and acceptor emission were observed in the 5 μM (8.2% and 7.8% respectively) and 20 μM samples (15.1% and 8.4% respectively).

Spectral overlap between donor, acceptor, and photochromic nucleotide(s) is vital to FRET and excitonic transfer and thus was appropriately engineered in this exemplary switch to achieve maximum modulation of both excitonic flow and photon emission as shown in Example 4.

For the exemplary all-optical excitonic switch, resonant energy transfer can occur either from donor to acceptor or from donor to photochromic nucleotide(s), which act as an alternate set of acceptor(s). FRET efficiency and excitonic transfer depend upon the properties of all chromophores involved in their respective transfer pathways. The properties that govern the relative FRET efficiency and excitonic transfer in the ON vs. OFF states are the inter-chromophore distances, spectral overlap of donor emission and acceptor absorption, and the relative orientations of the donor emission and acceptor absorption dipole moments. The self-assembly of the exemplary all-optical excitonic switch device is illustrated in FIG. 4C, wherein all positions (and hence inter-chromophore distances) are precisely controlled through sequence design (Table 4). The spectral overlap of both donor and acceptor along with donor and photochromic nucleotide are appropriately engineered in our design to achieve maximum modulation of both excitonic flow and photon emission (FIG. 3A). However, the chromophores used in this Example have been attached to the ssDNA via single, flexible tethers; thus, achieving complete control over the relative dipole orientations of the optical switching elements is not possible.

For clarity in visualizing spectral overlap, normalized absorption (dashed curves) and emission (solid curves) spectra of the photochromic strand with three photochromic units, ATTO 390 donor strand, and ALEXA 488 acceptor strand used in this Example are shown in FIG. 4D, FIG. 4E, and FIG. 4F, respectively. Unaltered absorbance spectra, also relevant to the absolute modulation efficiency, are provided in the FIG. 3B.

The large absorbance below 300 nm is due solely to the DNA strands. The photochromic nucleotide can be cycled from the open non-visible light absorbing configuration (ON state—red dashed/dotted curve, FIG. 4D) to the closed visible light absorbing configuration (OFF state—red dashed curve, FIG. 4D) by exposure to 300 nm UV light, while cyclo-reversion is accomplished by exposing it to 455 nm visible (VIS) light.

These wavelengths were chosen in order to: i) minimize damage to the DNA, which is known to occur with UV light of shorter wavelengths and ii) utilize wavelengths as close as possible to the absorption maxima of the photochromic nucleotide. To minimize perturbation of the photochromic nucleotide during optical measurements, the donor was excited with 350 nm visible light (FIG. 4E, donor absorption spectrum, blue dashed curve), resulting in donor emission (blue solid curve) with a maximum at 460 nm. The acceptor's absorption spectrum (FIG. 4F, green dashed curve) overlaps the donor emission, resulting in FRET-based acceptor emission (green solid curve) at 520 nm.

While synthesis of a reversible photochromic moiety covalently bound to a nucleotide and incorporated into a single strand of DNA (ssDNA) (FIG. 4D chemical structure insets) has been previously demonstrated, integrating multiple diarylethene units into ssDNA, which was required to examine the selectivity of the all-optical excitonic switch in this specific situation, has remained elusive. The increased absorbance of one versus three photochromic nucleotides per strand is expected to produce a threefold increase in the donor-acceptor emission modulation as shown in FIG. 5A-FIG. 5D. The relative absorbance of one versus three photochromic nucleotides (FIG. 4G) indicates this assumption holds true, with the corresponding ON and OFF emission data shown in FIG. 4H.

FIG. 4A-FIG. 4H show an exemplary all-optical excitonic switch and its characteristics. FIG. 4A and FIG. 4B show schematic of the all-optical excitonic switch with one and three photochromic nucleotides, respectively. FIG. 4C shows schematic of the self-assembly process allowing for precisely controlled placement of the optical components employed in the all-optical excitonic switch through binding domains. FIG. 4D shows the normalized absorption spectra of the photochromic strands in the CLOSED (absorbing) and OPEN (non-absorbing) configurations. Schematic insets illustrate molecular configuration changes occurring in the photochromic nucleotides with the ring system formed or opened highlighted in red. FIG. 4E and FIG. 4F show normalized absorption and emission spectra of the donor and acceptor strands, respectively, utilized in this Example. FIG. 4G shows the relative absorbance spectra of photochromic strands containing the single specific photochromic nucleotide versus three specific photochromic nucleotides, illustrating the three-fold increase in absorption. FIG. 4H shows the static emission plots of the all-optical excitonic switch in the ON and OFF state collected using the two design schemes as shown in FIG. 4A and FIG. 4B.

This Example shows when the triple photochromic strand (lower plot) was employed, the excitonic modulation of an all-optical switch was improved.

The all-optical switches disclosed herein can comprise a series of photochromic nucleotides assembled into the backbone of DNA oligonucleotides by chemical synthesis. Like natural nucleotides, these photochromic nucleotides comprise a deoxyribose sugar, a phosphate group, and a heterocyclic base capable of pairing with complementary nucleotides.

What makes the photochromic nucleotides from natural ones is that the bases of these photochromic nucleotides are covalently modified to be part of a diarylethene photochromic unit. Diarylethenes, originally developed for optical information storage purposes, undergo a reversible photoinduced electro-cyclic ring closure reaction. Ultraviolet (UV) irradiation forms a colored closed-ring isomer, while irradiation at longer (visible) wavelengths leads to a colorless open-ring form, thereby rendering the nucleotide absorptive or transmissive to light of specific wavelengths, a property that is critical for the all-optical switches disclosed herein.

Depending on the chemical nature of the diarylethenes, switching can be fully reversible, and more than 10⁵ cycles have been reported for some systems. Using different diarylethenes, together with using different donor(s), acceptor(s), DNA duplex, numbers of photochromic nucleotides comprising diarylethene, its analog, or mixture thereof and adjusting distances among donor, acceptor, and photochromic nucleotide(s), leads to different all-optical switches disclosed herein.

### Example 7

Characterizing the Exemplary All-Optical Excitonic Switch by Fluorescence Measurements

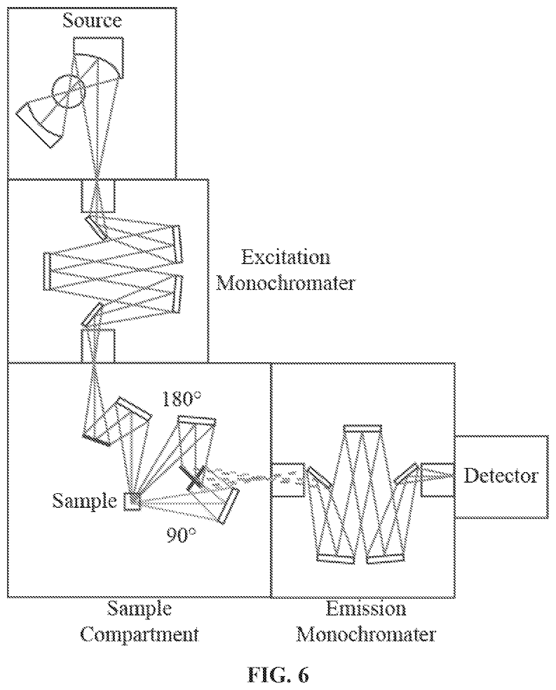

Optical characterizations involving acquisition of both the absorbance and fluorescence spectra of an exemplary all-optical excitonic switch were performed in this Example. UV-Vis absorbance spectra were collected for each individual strand using a dual-beam Cary 5000 UV-Vis-NIR spectrophotometer (Agilent Technologies) by pipetting 50 μL of sample into a black-mask, 1 cm path length, low head space, sub-micro quartz cuvette (Cat. #26.LHS-Q-10/Z20, Starna Cells). Static and dynamic emission spectra were collected using a Fluorolog-3 spectrofluorometer (HORIBA Scientific). Fluorescence emission detector geometries are illustrated in FIG. 6.

FIG. 6 shows the detector geometry for acquisition of all-optical excitonic emission spectra. For the liquid environment measurements, a perpendicular excitation light source interacts with the sample via a 2 mm by 2 mm window on the source side of the cuvette. The fluorescence emission is transmitted through a 2 mm by 10 mm window on the detector side of the cuvette (cyan lines) and detected via a photomultiplier tube (PMT, detector) aligned at 90° relative to the excitation. For the solid environment measurements, the excitation light source remained perpendicular to the sample, but the detector samples fluorescence emission aligned ˜180° relative to the sample (purple lines). The solid alignment geometry is required because very little fluorescence emission is delivered in the direction parallel to the quartz slide.

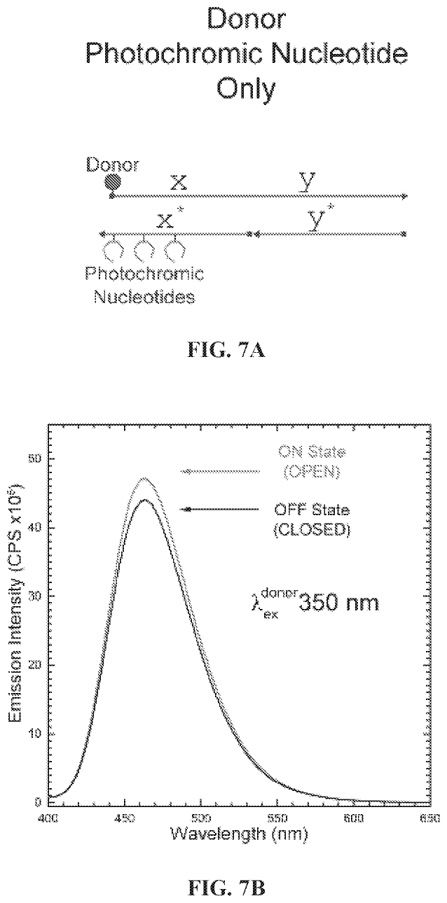

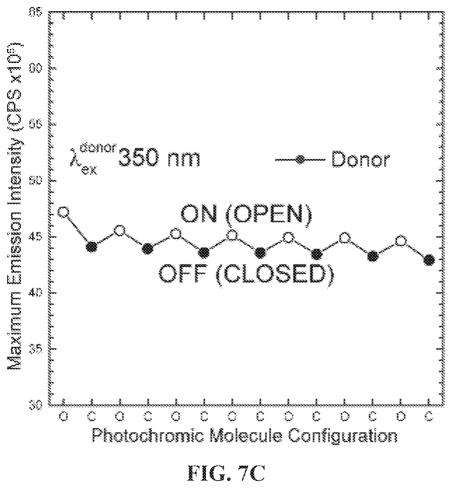

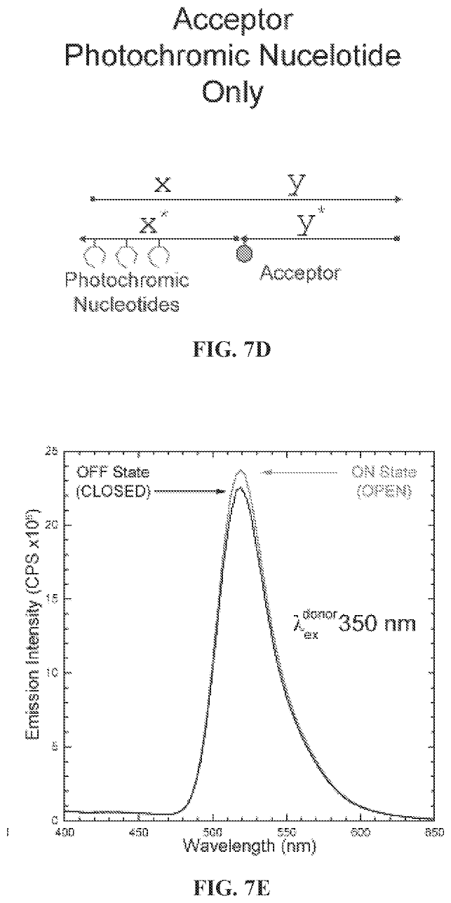

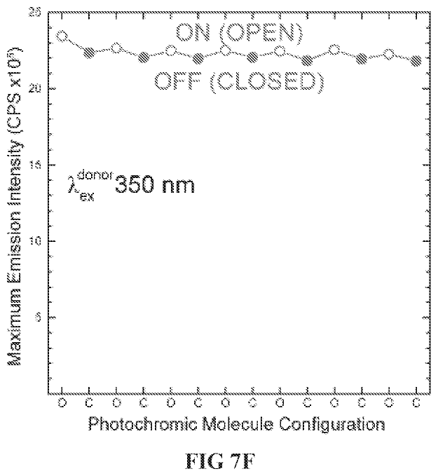

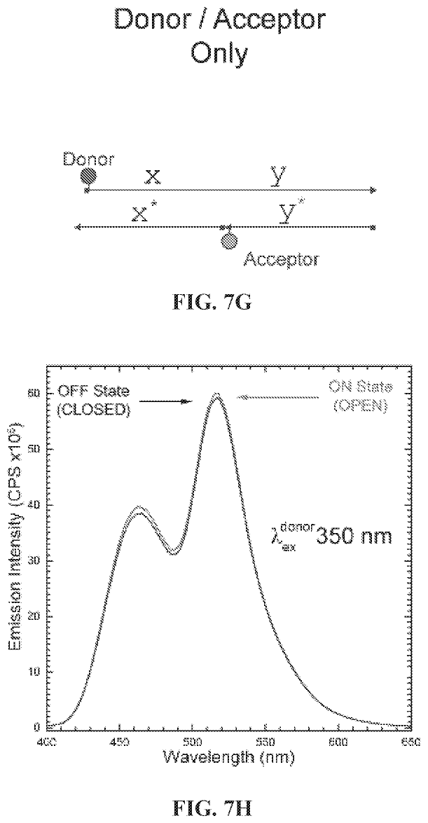

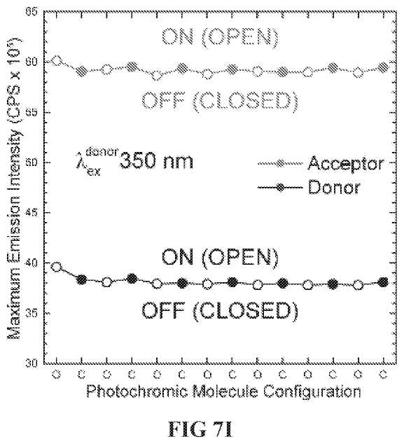

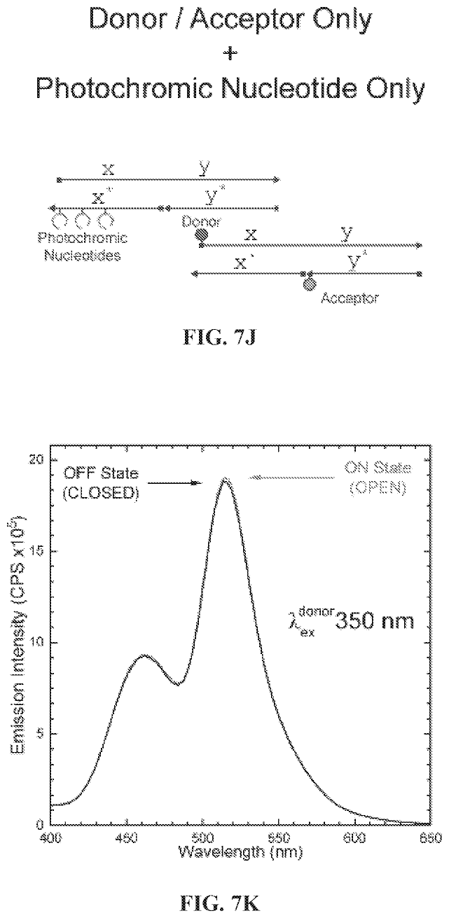

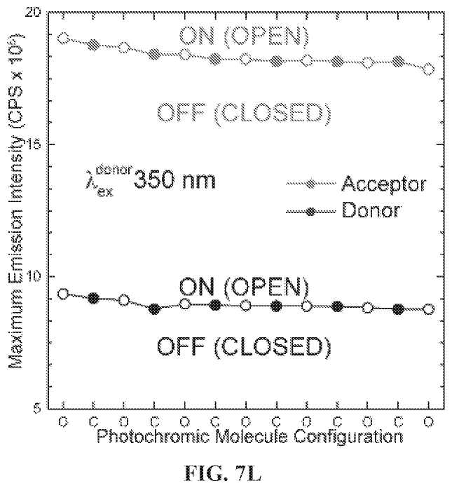

To better understand the interaction of the photochromic nucleotides with the donor and acceptor chromophores, a series of control experiments were performed. In these control experiments, specific DNA strands of the all-optical excitonic switch were replaced with bare ssDNA strands as illustrated in FIG. 7A, FIG. 7D, FIG. 7G, and FIG. 7J.

Liquid phase data were obtained by pipetting 50 μL of the all-optical excitonic switch solution into a black-mask, 1 cm path length, sub-micro fluorometer quartz cuvette (Cat. #16.40F-Q-10/Z15, Starna Cells). Solid phase data were obtained by pipetting 5 μL of the all-optical excitonic switch solution onto a fused quartz microscope slide (Chemglass Life Sciences, USA) and placing it in a custom-built vacuum chamber at 0.24 Torr for twelve hours. Quartz slides were cleaned prior to sample deposition by sonication in 2% Hellmanex (Hellma Analytics) solution for 5 minutes, followed by sonication in ultrapure water for 5 minutes and drying with ultra-high purity nitrogen (UHP, 99.999%).

FIG. 7A-FIG. 7L show all-optical excitonic switch controls performed at a 5 μM DNA concentration in the liquid environment. FIG. 7A-FIG. 7C show that a donor in a DNA duplex with three photochromic nucleotides hybridized but no acceptor present produced slight modulation of the donor emission, indicating excitonic absorption and FRET transfer interruption. FIG. 7D-FIG. 7F show that an acceptor in a duplex with three photochromic molecules hybridized but no donor present produced slight acceptor emission modulation demonstrating excitonic absorption and FRET transfer interruption. FIG. 7G-FIG. 7I show that a donor/acceptor in a duplex with no photochromic nucleotides hybridized produced no significant modulation of either the donor or acceptor emission curve, signifying the excitonic absorption and FRET transfer interruption was directly attributable to the presence of the photochromic nucleotides. FIG. 7J-FIG. 7L show that a donor/acceptor dyes hybridized with three photochromic nucleotides hybridized to a bare all-excitonic construct outside the Förster radius produced negligible modulation. All data presented without normalization for concentration nor (in the case of FIG. 7K and FIG. 7L) for dilution.

When the acceptor dye was omitted from the all-optical excitonic switch, as shown in FIG. 7A-FIG. 7C, it was hypothesized that modulation of the donor by the photochromic nucleotides via absorption, FRET, or both processes should occur. As shown in FIG. 3, the photochromic nucleotides in the closed absorbing configuration, (red dashed curve), can interact with both the donor absorbance (blue dashed curve) and emission (blue solid curve). FIG. 7B and FIG. 7C demonstrate some emission intensity modulation, indicating the photochromic nucleotides absorb excitonic energy and interrupt FRET transfer of the donor emission. It could be therefore concluded that the saw-tooth plot shown in FIG. 7C validates our excitonic energy absorption hypothesis, which in turn causes FRET transfer modulation. Likewise, when the donor dye was omitted from the all-optical excitonic switch, as shown in FIG. 7D-FIG. 7F, it can be hypothesized that the modulation of the acceptor by the photochromic nucleotides via absorption, FRET, or both should occur. As shown in FIG. 3, the photochromic nucleotides in the closed absorbing configuration (red dashed curve) can interact with both the acceptor absorbance (green dashed curve) and emission (green solid curve) as shown in FIG. 7E-FIG. 7F demonstrate some emission intensity modulation, confirming the hypothesis that the photochromic nucleotides also absorb excitonic energy and interrupt FRET transfer to the acceptor dye. It can be further hypothesized that no modulation would be observed for the following controls: when photochromic nucleotides were not included in the switch construct as shown in FIG. 7G or when both the donor and the acceptor were excluded from the switch construct (i.e., only photochromic nucleotides present) and mixed in solution with a switch construct with only the donor and acceptor (i.e., no photochromic nucleotide; FIG. 7J). For the latter control (FIG. 7J), it was assumed that no near field interaction, and thus no modulation, would occur between the photochromic nucleotides-only switch construct and the donor/acceptor pair-only construct. Both negative controls, as shown in FIG. 7H-FIG. 7I and FIG. 7K-FIG. 7L, demonstrate virtually no modulation took place when cycling the all-optical excitonic switch controls, thus substantiating our hypotheses. The data shown in FIG. 7I did not show saw-tooth behavior and thus suggested that modulation via excitonic absorption or FRET interruption was not possible without the photochromic nucleotides present. FIG. 7L did not show saw-tooth behavior and this absence of modulation reinforced the need for direct, distance dependent interaction of the photochromic nucleotides with the donor/acceptor pair to produce excitonic absorption and FRET interruption. It was noted that the data shown in FIG. 7K did indicate some inner filter effect on the donor emission in the presence of the photochromic nucleotides-only switch; this can be seen by comparing the donor emission in FIG. 7H and FIG. 7K. The control experiments indicated that there were a variety of potential pathways for energy transfer, including but not limited to using different photochromic nucleotides, donor/acceptor pair, DNA duplexes, and number of the photochromic nucleotides.

Static and dynamic optical measurements were performed to assess FRET and excitonic emission modulation of the exemplary all-optical switch by monitoring changes in both the donor and acceptor emission with the photochromic nucleotides in either their open (ON) or closed (OFF) configuration.

For static measurements, the photochromic nucleotides were set to either an open or closed configuration. Conversely, dynamic measurements required the simultaneous light exposure of both the photochromic nucleotides (to initiate opening or closing) and excitation of the donor or acceptor while concurrently monitoring the donor or acceptor emission.

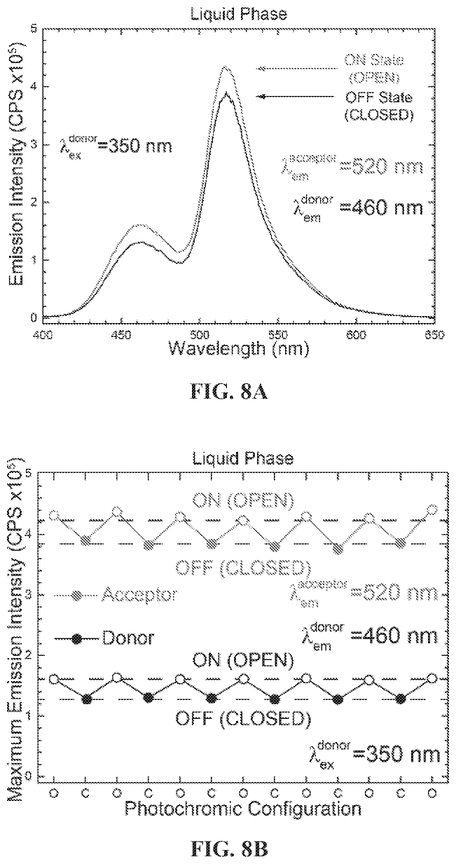

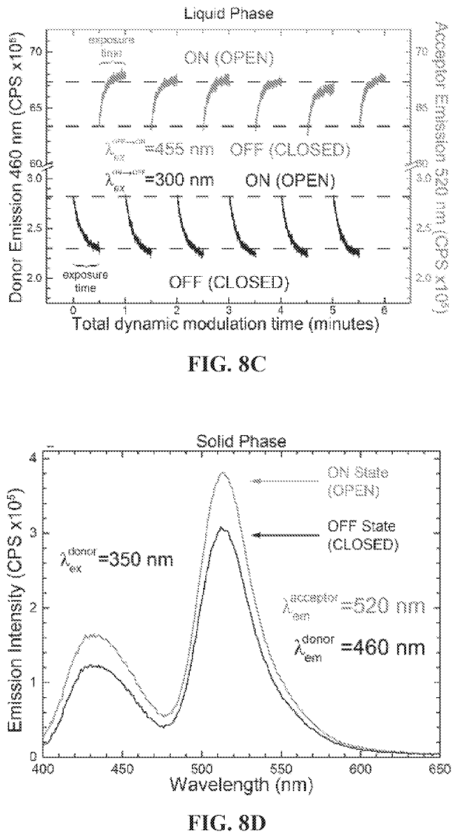

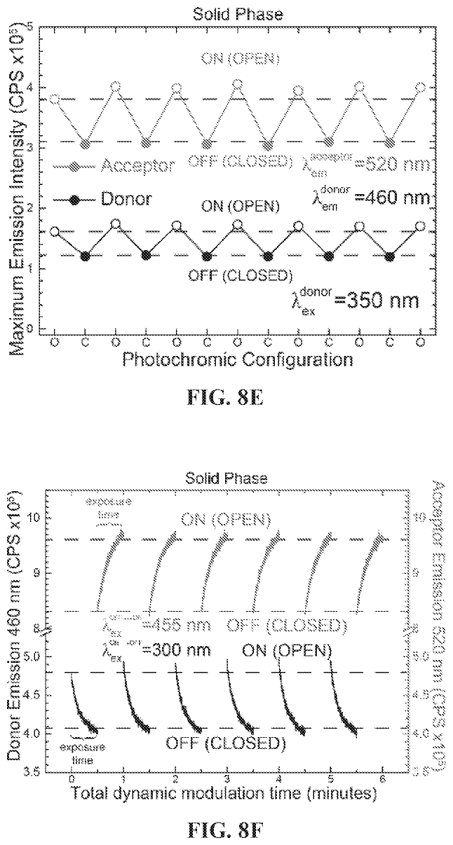

FIG. 8A-FIG. 8F show the switch emission of an exemplary all-optical excitonic switch with three photochromic nucleotides attached. FIG. 8A-FIG. 8C show the emission modulation data obtained for the all-optical excitonic switch operated at concentrations of 20 μM in the liquid. FIG. 8D-FIG. 8F show the emission modulation data obtained for the all-optical excitonic switch operated 52 mM (calculated from the method described below) in the solid phases with an exposure time of 30 seconds. FIG. 8A and FIG. 8D show the static emission plots of the all-optical excitonic switch in the ON and OFF states demonstrating modulation between the donor and the acceptor emission maxima. A blue shift in the solid phase donor emission is noted, maybe due to solvatochromic changes of the chromophores because of solidification from the liquid phase.

FIG. 8B-FIG. 8E show the saw-tooth plots demonstrating the changes in the donor and acceptor emission as the photochromic nucleotides are cycled between the open (O) and closed (C) configuration (i.e. ON and OFF states) six times, with near complete resetting of the all-optical excitonic switch between each cycle (i.e., no evidence of cyclic fatigue). Dashed lines have been added to aid in visualizing an ON/OFF threshold between the open and closed configuration of the photochromic nucleotide for both the donor and acceptor emission. FIG. 8C-FIG. 8F show the dynamic modulation emission plots of the donor and the acceptor strand as the photochromic nucleotides are cycled between the open and the closed configuration six times; exponential behavior indicates first-order reaction kinetics.

FIG. 8A and FIG. 8D show the static emission spectra of the donor and acceptor strands of the exemplary all-optical switch. The spectra were acquired by continuously exciting the donor strand. FIG. 8A and FIG. 8D indicate an overall decrease in the static emission scans of the all-optical excitonic switch in the liquid and the solid phase, respectively, when the photochromic nucleotides are set to the OFF state, indicating they act to inhibit the donor and acceptor emission and abate the FRET process. Subtle variations in the emission intensities and slight absorbance shifts are noted (FIG. 8D). Repeated cycling of the photochromic nucleotides between the open and closed configuration modulated the emission of both the donor and the acceptor (FIG. 8A and FIG. 8D). Comparing the maximum donor and acceptor emission after each opening and closing event resulted in saw-tooth behavior (FIG. 8B and FIG. 8E) with clear ON/OFF switching contrast indicative of modulation with minimal fluctuation. The static emission scans (FIG. 8A-FIG. 8D) and saw-tooth behavior (FIG. 8B-FIG. 8E) reinforce the hypothesis of modulation of FRET and excitonic emission as well as demonstrate consistent resetting of the photochromic nucleotides between the open and closed configurations, validating successful all-optical excitonic switching.

Dynamic measurements require the simultaneous light exposure of both the photochromic nucleotides (to initiate opening or closing) and excitation of the donor or acceptor while concurrently monitoring the donor or acceptor emission. Dynamic modulation scans as in FIG. 8C and FIG. 8F were performed by exposing the photochromic nucleotides with ultraviolet (UV) λₑₓ^{ON→OFF} 300 nm or visible (VIS) λₑₓ^{OFF→ON} 455 nm light while monitoring the donor or acceptor emission at 460 nm or 520 nm, respectively. Either the donor or the acceptor emission can be examined real-time while simultaneously opening or closing the photochromic nucleotides.

The ability to observe real-time changes in the donor or acceptor emission while simultaneously changing/cycling the configuration (open or closed) of the photochromic nucleotides (i.e., dynamic modulation emission scans) would provide insight into the dynamics of the all-optical excitonic switch (i.e., dynamics of the ON and OFF states). Accordingly, dynamic modulation scans in FIG. 8C and FIG. 8F were performed by exposing the photochromic nucleotides while monitoring the donor or acceptor emission. Time dependent changes in the donor and the acceptor emission provide a convenient proxy to monitor the changes from ON to OFF or OFF to ON states (i.e., fully versus partially ON or OFF) of the all-optical excitonic switch. From FIG. 8C and FIG. 8F, time dependent exponential growth/decay of the donor and acceptor emission is observed as the photochromic nucleotides are excited over an exposure time of 30 seconds.

From the exponential behavior of the dynamic modulation scans (FIG. 8C and FIG. 8F), rate constants can be extracted and used to predict the amplitude difference between the ON and OFF states of the saw-tooth plots (FIG. 8B and FIG. 8E). The amplitude difference between the ON and OFF states also provides a means for obtaining a well-defined characteristic switching time (Qs) based on knowledge of the incident light.

The exponential nature of the dynamic modulation behavior (FIG. 8C and FIG. 8F) provides a method to determine the mean time for modulation (τᵢ) occurring during the exposure time (tₑ) of the photochromic nucleotide and all-optical excitonic switch for two regimes: (i) τᵢ close (τ_{c}), the mean time to close the photochromic nucleotide when exposed at 300 nm, and (ii) τᵢ open (τₒ), the mean time to open the photochromic nucleotide when exposed at 455 nm. The sum of the two mean times for modulations, τₒ and τ_{c}, is defined as τ_{Σ}, the time to complete one cycle from the ON state to the OFF state and back to the ON state.

As both the static and dynamic modulation emission scans exhibit modulation and the dynamic modulation emission data exhibit exponential behavior, it was hypothesized that the changes in the maximum static emission intensities of the donor or acceptor (FIG. 8B and FIG. 8E), termed the switching amplitude (Δₛ), is related to a well-defined characteristic switching time (tₛ) of the all-optical excitonic switch. Assuming a first order reaction rate, the relationship between Δₛ and tₑ (see “Switching Time Derivation” and “Characteristic Switching Time Derivation” below) with only one modeling parameter was derived and the results demonstrates the relationship holds over a range of tₑ's with the ability to extract tₛ.

### Solid Phase Concentration Calculations

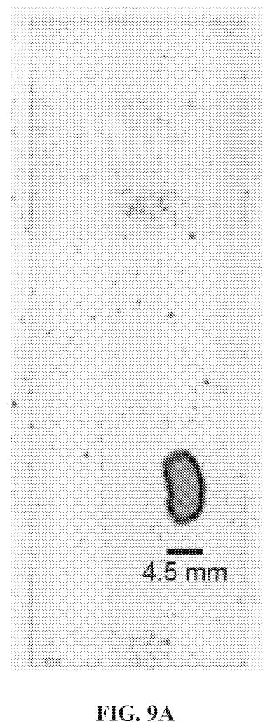

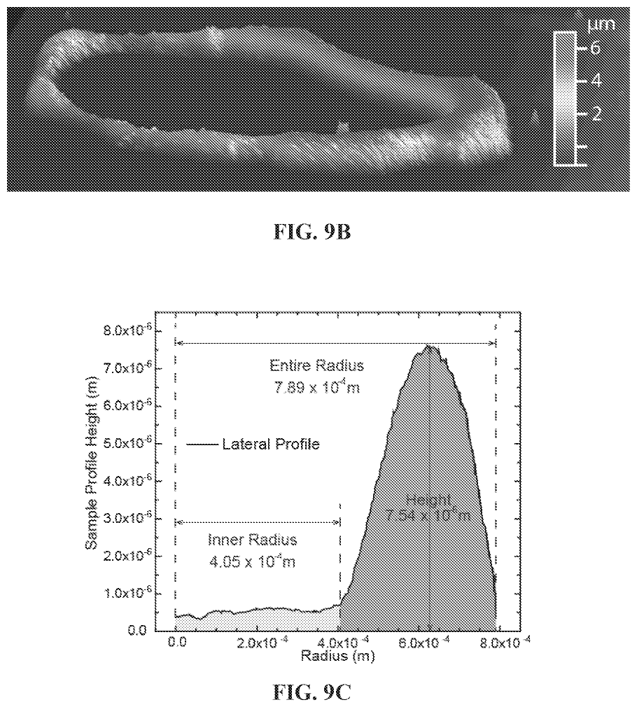

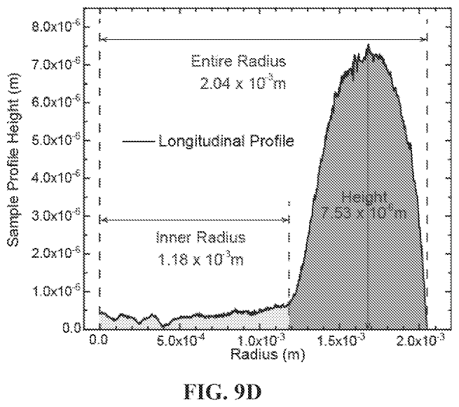

To determine the location of the all-optical excitonic switches in the solid phase sample, a fluorescence optical image of the solid phase sample using a ProteinSimple FluoroChem Q MultiImage III chemiluminescent imaging system running AlphaView software version 3.4.0.0. as shown in FIG. 9A was collected. Since a 534 nm excitation filter and a 606 nm emission filter was used, only the emission of the acceptor was collected. The grayscale image was reversed to enhance the contrast between the sample and the quartz slide, hence the emission is shown as black. The fluorescence image shows that the all-optical excitonic switches are located only in the outer elliptical ring of the solid phase sample.

To estimate the bulk concentration of the solid phase sample, profilometry measurements were performed using a Bruker Dektax XT-A Stylus profilometer. FIG. 9A shows the representative solid phase sample imaged using a 534 nm excitation filter and a 606 nm emission filter; the grayscale image was reversed for clarity. A clear elliptical ring on the outer periphery can be observed in the image indicating the all-optical excitonic switch migrates to the edges of the sample during desiccation. The elliptical ring dimensions were confined within the window size of the fluorometer excitation beam. FIG. 9B shows the 3D profilometer map of a representative solid phase sample where the elliptical ring was observed within the outer periphery of the sample.

The profilometry data were plotted in FIG. 9C and FIG. 9D and the resulting radial distances and profile height along the lateral and longitudinal ellipse directions were used to calculate the volume of the ellipse and, specifically, the volume of the outer elliptical ring. It was noted that most of the solid phase sample, and thus the all-optical excitonic switches, reside in the outer elliptical ring which correlates with the emission image (FIG. 9A) showing the location of the all-optical excitonic switches.

FIG. 9C and FIG. 9D indicate the profilometer data collected from the solid phase sample along the lateral and longitudinal directions of the ellipse. For each direction, the sample height and the inner and outer ellipse radii are indicated. Note that the profiles have been divided into the non-fluorescing region (light shading) and the fluorescing region (dark shading).

The data suggested that evaporation while the sample was desiccated initially occurred from the center of the sample and moved outward during the transition from the liquid to solid phase. Hence, very little of the solid phase was within the inner ellipse and was instead primarily located in the outer elliptical ring. The solid phase concentration of all-optical excitonic switches can then be determined from the volume of the solid phase in the outer elliptical ring using the following Equations (1)-(6):

Aᵢ=πr₁r₂ (1)

Aₑ=πr₃r₄ (2)

where r₁ and r₂ are the radii (major and minor) of the inner portion of the ellipse, r₃ and r₄ are the radii of the entire ellipse, and Aᵢ and Aₑ are the areas of the inner and entire ellipses, respectively. The area, Aᵣ, and volume, Vᵣ, of the outer elliptical ring are given by:

Aᵣ=Aₑ−Aᵢ, (3)

Vᵣ=AᵣHᵣ, (4)

where Hᵣ is the height of the outer elliptical ring. The concentration of the all-optical excitonic switches can be found using mass balance relationship:

CᵣVᵣ=c_{liq}V_{liq}, (5)

where Cᵣ is the concentration of the all-optical excitonic switches in the outer elliptical ring while C_{liq} and V_{liq} are the concentration and volume of the liquid phase solution pipetted on the slide, respectively.

Solving for the concentration of the all-optical excitonic switches, Cᵣ, gives:

$${C}_{r} = \frac{{C}_{liq} {V}_{liq}}{{V}_{r}} . \qquad ( 6 )$$

Using the profilometry data, Equation (6) was used to calculate the concentration of the all-optical excitonic switches in the solid phase and is listed in Table 5.

As a frame of reference (i.e., validity check) to Cᵣ determined via Equation (6) using the profilometry data, a theoretical approach was also used to estimate the concentration of all-optical excitonic switches in the solid phase by using the volume of the double stranded DNA (dsDNA) scaffold, V_{dsDNA}, given by:

V_{dsDNA}=πr²l, (7)

where the r and l are the radius and length of the dsDNA, respectively. The concentration of all-optical excitonic switches, C_{r,theory}, is determined by:

$${C}_{r , theory} = \frac{1 switch }{{V}_{dsDNA}} \frac{mole}{Av \#} , \qquad ( 8 )$$

where Av # is Avogadro's number. Equation (8) was used to calculate the theoretical concentration of all-optical excitonic switches in the solid phase and is listed in Table 5 to compare to that calculated by Equation (6). The comparison shows that the values are within an order of magnitude. Hence, the profilometry approach to determine the concentration of the all-optical excitonic switches in the solid phase is sound.

TABLE 5
Liquid and solid phase concentration values

| | | Liquid Phase | | Solid Phase |
|---|---|---|---|---|
| | Liquid Phase | Concentration, | Solid Phase | Concentration, |
| | Volume, | C_{liq} | Volume, | Cᵣ |
| | V_{liq} (L) | (M) | Vᵣ (L) | (M) |
| Profilometer | 5.0 × 10⁻⁶ | 20 × 10⁻⁶ | 8.5 × 10⁻⁹ | 16 × 10⁻³ |
| Theoretical | | | | 52 × 10⁻³ |

### Switching Time Derivation

Assuming a first order reaction rate for the all-optical switch's opening and closing upon exposure to specific wavelengths of light, the relationship between Δₛ and tₑ is:

$${Δ}_{s} = S [ \frac{( 1 - {e}^{- {t}_{e} / {τ}_{o}} ) ( 1 - {e}^{- {t}_{e} / {τ}_{c}} )}{1 - {e}^{- ( {τ}_{o} + {τ}_{c} ) - {t}_{e} / {τ}_{o} {τ}_{c}}} ] \qquad ( 9 )$$

where τₒ and τ_{c} are the mean time to modulate from closed to open, and the mean time to modulate from open to closed, respectively, and S is a single fitting offset representative of the steady state equilibrium (see below for complete derivation). The simple elegance of Equation (9) allows Δₛ versus tₑ data to be modeled with a single parameter, S. The validity of the hypothesis is then tested by fitting Δₛ versus tₑ data using Equation (9).

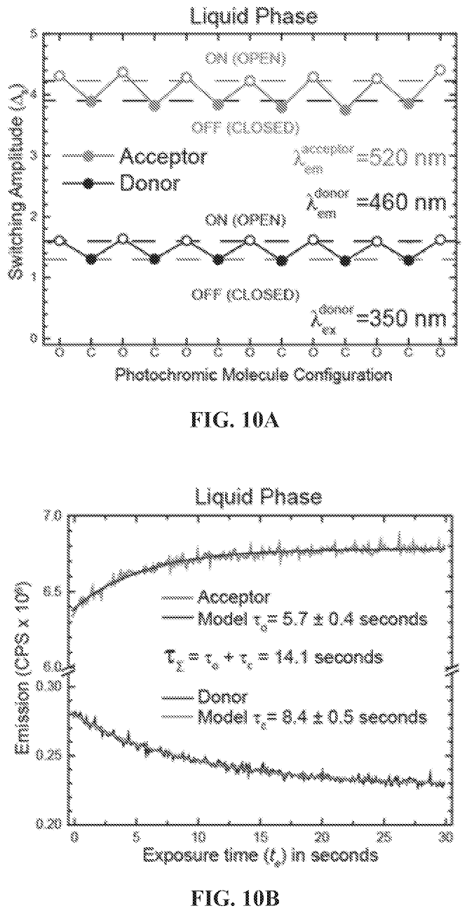

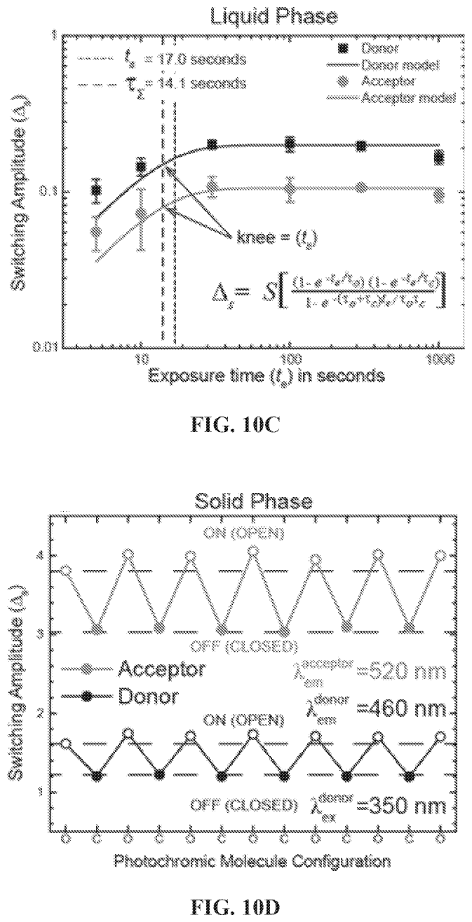

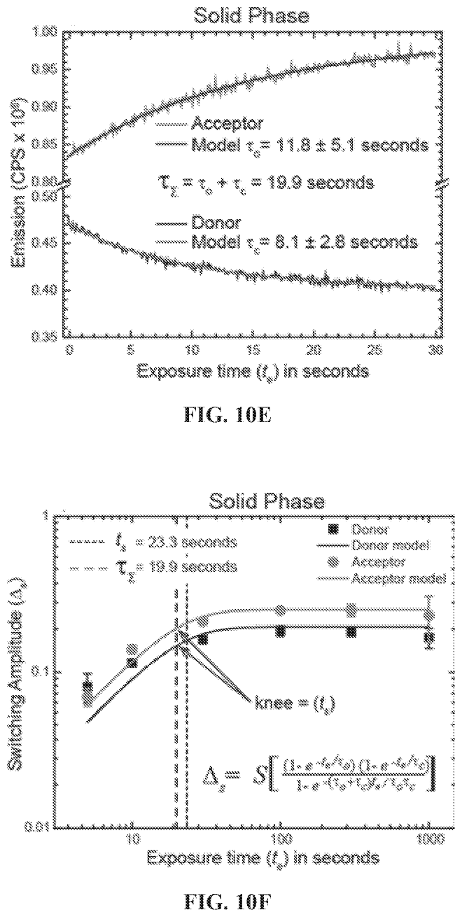

Obtained from the static emission scans, Δₛ of the donor or acceptor is defined as the fractional difference between the maximum emission intensities in the ON or OFF state of either the donor or the acceptor, and is given by:

$${Δ}_{s} = \frac{{I}_{open} - {I}_{close}}{{I}_{open}} , \qquad ( 10 )$$

where Iₒₚₑₙ and I_{closed} are the maximum donor (462 nm liquid and 433 nm solid) or acceptor (517 nm liquid and 513 solid) emission intensities when the photochromic nucleotides are in the open configuration or closed configuration, respectively. For each exposure time, Δₛ was determined and plotted as a function of the state (ON versus OFF) as shown in FIG. 10A.

Assuming first order reaction kinetics behavior for ring opening and closing of the photochromic nucleotides, the dynamic modulation emission data (FIG. 10C and FIG. 10F) can be fit with an exponential function described by:

I=Iₒe^{-t}^{ₑ}^{/τ}^{ᵢ}+I_{offset}, (11)

where I, I₀, tₑ, and τᵢ are the final emission intensity of the donor or acceptor, the initial emission intensity of the donor or acceptor, the photochromic nucleotides' exposure time, and the mean time for modulation (time to open, τᵢ=τₒ, or close, τᵢ=τ_{c}, the photochromic nucleotides), respectively. I_{offset}, establishes the donor or acceptor emission intensity offset from a zero baseline. From Equation (11), values for τᵢ may be extracted for both the closed to open cycle (τₒ) and open to closed cycle (τ_{c}), and their sum (τ_{Σ}) should theoretically be the same as tₛ, the characteristic switching time shown below.

### Characteristic Switching Time Derivation

Here an expression for the switching amplitude as a function of time for the all-optical excitonic switch is obtained.

Let [O] denote the concentration of open photochromic nucleotides and let [C] denote the concentration of closed photochromic nucleotides, then:

[O]+[C]=[S], (12)

where [S] is the total concentration of photochromic nucleotides.

Let Iₒ denote the photon flux of the light source used to open the photochromic nucleotides and let σₒ be the absorbance cross-sectional area for photochromic nucleotides opening. Then, the equation for the rate with which closed photochromic nucleotides are being lost from the sample due to conversion into open photochromic nucleotides is:

$$\frac{d [ C ]}{d t} = - {σ}_{o} {I}_{o} [ C ] \qquad ( 13 )$$

Similarly, let I_{c} be the photon flux of the light source used to close the photochromic nucleotides and let σ_{c} be the absorbance cross-sectional area for photochromic nucleotides closing. The rate with which the open photochromic nucleotides are being removed from the sample by conversion into closed photochromic nucleotides is:

$$\frac{d [ O ]}{d t} = - {σ}_{c} {I}_{c} [ O ] \qquad ( 14 )$$

It is useful to introduce the switching rates (γᵢ) and switching times (tᵢ) for opening (i=o) and closing (i=c) the hotochromic nucleotides according to:

$$\begin{matrix}{γ}_{0} = \frac{1}{{t}_{o}} = {σ}_{o} {I}_{o} , \\ and & ( 15 ) \\ {γ}_{c} = \frac{1}{{t}_{c}} = {σ}_{c} {I}_{c} . & ( 16 )\end{matrix}$$

### Switching Equations

The case when the all-optical excitonic switch is being cycled back and forth between the open and closed configuration is considered here. Consider a sequence of exposure times tₙ labeled by successive integers n, where n=0, 1, 2, 3, . . . , at which cycling from one configuration to the other is initiated. During the times t for which t₂ₙ<t<t₂ₙ₊₁ the photochromic nucleotides are being exposed with light that opens the photochromic nucleotides. Hence, from Equations (13) and (15) one has:

$$\frac{d [ C ]}{d t} = - {γ}_{o} [ C ] , when {t}_{2 n} < t < {t}_{2 n + 1} \qquad ( 17 )$$

During the times t for which t₂ₙ₊₁<t<t₂ₙ₊₂, the photochromic nucleotides are being exposed with light that closes the photochromic nucleotides. Hence, from Equations (14) and (16), one obtains:

$$\frac{d [ O ]}{d t} = - {γ}_{c} [ O ] , when {t}_{2 n + 1} < t < {t}_{2 n + 2} \qquad ( 18 )$$

Integrating Equation (17) within the exposure time interval t₂ₙ<t<t₂ₙ₊₁ gives:

[C]=[C]ₜ_{₂ₙ}e^{-γ(t-t}^{₂ₙ}⁾,for t₂ₙ<t<t₂ₙ₊₁ (19)

Integrating Equation (18) within the exposure time interval t₂ₙ₊₁<t<t₂ₙ₊₂ gives:

[O]=[O]ₜ_{₂ₙ}e^{-γc(t-t}^{₂ₙ₊₁}⁾,for t₂ₙ₊₁<t<t₂ₙ₊₂ (20)

Evaluating the concentrations at the end of the exposure time intervals, Equations (19) and (20) yield:

[C]ₜ_{₂ₙ₊₁}=[C]ₜ_{₂ₙ}e^{-γ}^{ₒ}^{(t}^{₂ₙ₊₁}^{-t}^{₂ₙ}⁾, (21)

[O]ₜ_{₂ₙ₊₂}=[O]ₜ_{₂ₙ₊₁}e^{-γ}^{_{c}}^{(t}^{₂ₙ₊₂}^{-t}^{₂ₙ₊₁}⁾, (22)

We now take all the exposure time intervals to be the same, that is:

tₙ₊₁−tₙ=tₑ for alln (23)

Hence, Equations (21) and (22) become:

[C]ₜ_{₂ₙ₊₁}=[C]ₜ_{₂ₙ}e^{-γ}^{ₒ}^{t}^{ₑ}, (24)

[O]ₜ_{₂ₙ₊₂}=[O]ₜ_{₂ₙ₊₁}e^{-γ}^{_{c}}^{t}^{ₑ} (25)

Using Equation (12), this last equation yields:

[S]−[C]ₜ_{₂ₙ₊₂}=([S]−[C]ₜ_{₂ₙ₊₁})e^{-γ}^{_{c}}^{t}^{ₑ} (26)

This can be rearranged to give:

[C]ₜ_{₂ₙ₊₂}=[S](1−e^{-γ}^{_{c}}^{t}^{ₑ})+[C]ₜ_{₂ₙ₊₁}e^{-γ}^{_{c}}^{t}^{ₑ} (27)

Using Equation (21) this becomes:

[C]ₜ_{₂ₙ₊₂}=[S](1−e^{-γ}^{_{c}}^{t}^{ₑ})+[C]ₜ_{₂ₙ}e^{-(γ}^{ₒ}^{+γ}^{_{c}}^{)t}^{ₑ} (28)

When cycling back and forth for over a long period of time, steady state is achieved in which:

[C]ₜ_{₂ₙ₊₂}=[C]ₜ_{ₙ},for alln (29)

and similarly for the [O] concentration. Hence, Equation (28) becomes:

[C]ₜ_{₂ₙ}=[S](1−e^{-γ}^{_{c}}^{t}^{ₑ})+[C]ₜ_{₂ₙ}e^{-(γ}^{ₒ}^{+γ}^{_{c}}^{)t}^{ₑ} (30)

Solving this equation for [C]ₜ_{₂ₙ} yields the steady-state value:

$${[ C ]}_{{t}_{2 n}} = [ S ] \frac{1 - {e}^{- {γ}_{c} {t}_{e}}}{1 - {e}^{- ( {γ}_{o} + {γ}_{c} ) {t}_{e}}} \qquad ( 31 )$$

Substituting Equation (31) into Equation (24) yields the following expression for the steady state value of [C]ₜ_{₂ₙ₊₁}:

$${[ C ]}_{{t}_{2 n + 1}} = [ S ] \frac{{e}^{- {γ}_{o} {t}_{e}} ( 1 - {e}^{- {γ}_{c} {t}_{e}} )}{1 - {e}^{- ( {γ}_{o} + {γ}_{c} ) {t}_{e}}} \qquad ( 32 )$$

The switching amplitude Δₛ is the difference, as measured by emission, between the concentration of closed photochromic nucleotides with respect to the former exposure time and the most recent exposure time given by:

Δₛ=[C]ₜ_{₂ₙ}−[C]ₜ_{₂ₙ₊₁} (33)

Substituting Equations (31) and (32) into Equation (33) gives:

$${Δ}_{s} = [ S ] \frac{( 1 - {e}^{- {γ}_{o} {t}_{e}} ) ( 1 - {e}^{- {γ}_{c} {t}_{e}} )}{1 - {e}^{- ( {γ}_{o} + {γ}_{c} ) {t}_{e}}} \qquad ( 34 )$$

In terms of the exposure times, Equation 34 can be written as

$${Δ}_{s} = [ S ] \frac{( 1 - {e}^{- {t}_{e} / {t}_{o}} ) ( 1 - {e}^{- {t}_{e} / {t}_{c}} )}{1 - {e}^{- ( {t}_{o} + {t}_{c} ) / {t}_{o} {t}_{c}}} \qquad ( 35 )$$

In the limit of very large exposure times, tₑ, Δₛ no longer changes because it can be assumed that all photochromic nucleotides are either in the closed or open configuration. Hence, can be written as:

$${lim}_{Δ t \to ∞} {Δ}_{s} = [ S ] \qquad ( 36 )$$

that is, if the exposure time is long enough, all the photochromic nucleotides are cycled from one configuration to the other. Taylor expanding Equation (34) for small tₑ, one has, to linear order:

$${Δ}_{s} = \frac{{γ}_{o} {γ}_{c}}{{γ}_{o} + {γ}_{c}} [ S ] {t}_{e} \qquad ( 37 )$$

or, substituting Equations (15) and (16), gives:

$${Δ}_{s} = \frac{1}{{t}_{o} + {t}_{c}} [ S ] {t}_{e} \qquad ( 38 )$$

At the point in which the limit of long exposure times first holds, that is, Δₛ first becomes constant (does not vary) and Equation (36) holds, then Equation (35) and Equation (36) must be equivalent hence:

$$\frac{1}{{t}_{o} + {t}_{c}} [ S ] {t}_{e} = [ S ] \qquad ( 39 )$$

The particular tₑ at which Δₛ first becomes constant is defined as the characteristic switching time, tₛ. Thus, Equation (39) should be written as:

$$\frac{1}{{t}_{o} + {t}_{c}} [ S ] {t}_{s} = [ S ] \qquad ( 40 )$$

In order for Equation (38) and Equation (36) to be equivalent, tₛ and the sum of tₒ and t_{c} (i.e., τ_{Σ}) must be the same. That is:

$${t}_{s} = \frac{{γ}_{o} + {γ}_{c}}{{γ}_{o} {γ}_{c}} = {t}_{o} + {t}_{c} = {τ}_{Σ} . \qquad ( 41 )$$

The value of tₛ can be determined from a plot of Δₛ versus tₑ data in the following approach. The point of intersection between a vertical line, drawn from the x-axis, and Δₛ versus tₑ data at the time at which Δₛ first shows non-varying, or constant, behavior provides the value of tₛ. This approach is shown in FIG. 10C and FIG. 10F.

### Time Trial Design

For the time trial experiments, the photochromic nucleotides were exposed for cycle times (t) of 1 second, 3 seconds, 5 seconds, 10 seconds, 30 seconds, 100 seconds, 300 seconds, and 1000 seconds as shown in Table 6. Each cycle of the time trial was performed as a series of four sequential excitation/emission scans whereby i) the all-optical excitonic switch was initially set to the ON state and a static emission scan (400 nm to 650 nm) was collected while exciting the donor of the all-optical excitonic switch with 350 nm VIS light, ii) the photochromic nucleotides were exposed to 300 nm UV light (τ_{c}) while simultaneously collecting donor emission (460 nm), iii) a static emission scan (400 nm to 650 nm) was collected while exciting the donor of the all-optical excitonic switch with 350 nm VIS light, and iv) the photochromic nucleotides were exposed to 455 nm VIS light (τₒ) while simultaneously collecting acceptor emission (520 nm). Note that fitting of very short time scales (1, 3, and 5 seconds) is questionable due to the difficulty of fitting segments too short to observe curvature.

### Absorbance Cross Section

The absorbance cross-section (σ) of the all-optical excitonic switch may be calculated by using the photon flux (J) determined in the previous section and the extracted mean times for modulation shown in Table S6 as follows. Let P denote the probability that a given photochromic nucleotide is switched. The time rate of change of P is given by:

$$\frac{d P}{d t} = σ J ( 1 - P ) \qquad ( 42 )$$

The general solution to this differential equation is:

P=1−exp^{-(σJt)} (43)

Using the boundary conditions, the initial condition is P=0 at (t=0), and the final condition is P=1 at (t=∞). For the final condition, it is assumed that, if one waits long enough, all the photochromic nucleotides will have switched. Let

$${t}_{( \frac{1}{2} )}$$

denote the exposure time needed to cause half the photochromic nucleotides to cycle; at this time P=½. Substituting these two quantities in Equation (43) and solving for σ, one has:

$$σ = \frac{ln ( 2 )}{J {t}_{( 1 / 2 )}} . \qquad ( 44 )$$

### Photon Energy, Photon Fluence, and Photon Flux

Photon energy (E) was calculated for the two wavelengths utilized to cycle the configuration of the photochromic nucleotide using:

$$E = \frac{h c}{λ} , \qquad ( 45 )$$

where h is Planck's constant, c is the speed of light, and λ is the wavelength of interest. For this work, 300 nm light was used to switch the photochromic nucleotide between the open (ON) to closed (OFF) configuration (o-c) and 455 nm light was used to switch the photochromic nucleotide between the closed (OFF) to open (ON) configuration (c-o). Using Equation 45, the photon energy at 300 nm (o-c) is 6.62×10⁻¹⁹ Joules and at 455 nm (c-o) is 4.37×10⁻¹⁹ Joules.

The number of photons per second (photon fluence) is calculated with:

$$H = \frac{W}{E} , \qquad ( 46 )$$

where H is the photon fluence, W is the measured power and E is photon energy at 300 nm or 455 nm respectively. Power data collected from the Fluorolog-3 spectrofluorometer using a LABMAX_TOP (Coherent Inc.) power meter (Model 1104622) coupled to a silicon diode photodetector (PM 30) was found to be 60×10⁻⁶ W at 300 nm and 150×10⁻⁶ W at 455 nm. Using Equation 46 the photon fluence is 9.1×10¹³ photons/sec at 300 nm and 3.4×10¹⁴ photons/sec at 455 nm.

The photon flux (J) (photons per unit area per second) is calculated with:

$$J = \frac{W}{E A} , \qquad ( 47 )$$

where A is the spot size area. Using a spot size of ˜4 mm², Equation 47 yields a photon flux of 2.3×10¹⁹ photons/(m²·s) at 300 nm and 8.6×10¹⁹ photons/(m²·s) at 455 nm respectively.

### Ultrafast Laser Switching

Assuming use of a typical UV-VIS optical parametric amplifier (OPA) pumped by a 1 kHz regenerative amplified ultrafast Ti:sapphire laser (outputting sub 100 fs pulses) we can calculate the photon flux using:

$$\frac{J}{pulse} = \frac{E λ}{h c A} , \qquad ( 48 )$$

where E is the energy per pulse, λ is the wavelength of light to cycle the photochromic nucleotide, h is Planck's constant, c is the speed of light, and A is the spot size. Assuming 10 μJ/pulse with a beam radius of 100 μm at the focused spot size area, Equation 48 yields a photon flux per pulse of 4.8×10²⁰ photons/m² at 300 nm and 7.3×10²⁰ photons/m² at 455 nm.

To determine the possibility of ultrafast switching of the photochromic nucleotide, we compared the photon flux produced by the Fluorolog-3 (see “Absorbance Cross Section”) in 30 seconds (see FIG. 10C and FIG. 10F) to the photon flux produced by a sub 100 fs pulse produced by a typical ultrafast laser system. Using Equation (47), the Horiba produces a photon flux of 6.8×10²⁰ photons/m² in 30 seconds at 300 nm, whereas Equation (48) yielded a photon flux of 4.8×10²⁰ photons/m² per pulse. This indicates the photon flux due to a single pulse from a typical ultrafast laser system should be sufficient to cycle the all-optical excitonic switch in the picosecond regime, with switching times limited by the intrinsic switching time of the photoswitch molecules rather than the ultrafast laser pulse duration.

The data in FIG. 8A-FIG. 8F was obtained with a tₑ of 30 seconds; therefore, only one tₑ can be used in Equation (11). To determine the validity of our hypothesis (i.e., Equation (9)), a set of time trial experiments were necessary and therefore performed over a range of specifically chosen tₑ's for the photochromic nucleotides shown below and tₛ was thereby obtained. For each tₑ of the time trails experiments, saw-tooth plots were generated as shown in FIG. 10A, and Δₛ of the donor and the acceptor emission were calculated using Equation (10).

Equation (11) was used to fit the dynamic modulation data from each of the time trial experiments. FIG. 10B and FIG. 10E show the fits of the averaged (6 data sets) dynamic modulation data for a photochromic nucleotide tₑ of 30 seconds for both the liquid and solid phase, respectively. Both τₒ and τ_{c} were extracted from the liquid and the solid phase averaged dynamic modulation data. A τₒ value of 5.7±0.4 (standard deviation) and 11.8±5.1 seconds was given for the liquid and solid phase, respectively, and a τ_{c} value of 8.4±0.5 and 8.1±2.8 seconds was given for the liquid and solid phase, respectively (see Table 6 for all time trial data). The complete time to cycle between the open and closed state (τ_{Σ}) is 14.1±0.9 seconds for the liquid phase and 19.9±7.8 seconds for the solid phase. FIG. 10B and FIG. 10E are representatives of all time trial dynamic modulation data collected.

FIG. 10A-FIG. 10F show the characteristic all-optical excitonic switch time. FIG. 10A and FIG. 10D show the characteristic switching time (tₛ) determination from the liquid and solid phase time trial experiments. FIG. 10B and FIG. 10E show the representative averaged saw-tooth plots extracted from 30 second exposure time liquid and solid phase time trial static emission scans. FIG. 10C and FIG. 10F show the representative dynamic modulation plots of the donor and acceptor emission by the photochromic nucleotides with exponential decay fits, extracted τₑ, τₒ, & τ_{Σ} times given. Switching amplitude (Δₛ) versus photochromic nucleotide exposure time (tₑ) of the photochromic nucleotides fitted with the first order reaction kinetics model (Eq. 9). Error bars were generated from averaged standard deviations of each time trial Δₛ series.

Plotting the switching amplitude (Δₛ) of the donor and acceptor emission collected from a series of time trial experiments as shown in FIG. 10A and FIG. 10D (See “Time Trial Design” Below) versus the photochromic nucleotide tₑ's in FIG. 10C and FIG. 10F enables modeling the time trial data using Equation (9). Because the dynamic modulation as in FIG. 9B and FIG. 9E exhibits first order reaction kinetics behavior from which τₒ and τ_{c} can be extracted, the switching amplitude (Δₛ) may be assumed to become constant if tₑ is sufficiently long; that is, if tₑ is long enough, all (i.e., a steady state amount) of the photochromic nucleotides are cycled from one configuration to the other. Theoretically, the characteristic switching time (tₛ) is then equivalent to τ_{Σ} (i.e., τₒ+τ_{c}) at the point when Δₛ becomes constant.

The data and the fits as shown in FIG. 10C and FIG. 10F (Eq. (9)) show the predicted linear increase in Δₛ in the range of ˜1-10 seconds, transitioning to a constant Δₛ between ˜20-1000 seconds where the transition between linear and constant is defined as the “knee”. The intersection of tangent lines (not shown) of the linear and constant regions of Δₛ gives tₛ (dashed line in FIG. 10C and FIG. 10F), where tₛ was found to be 17.0 and 23.3 seconds for the liquid and solid phases, respectively. The value of tₛ versus the calculated (from FIG. 10B and FIG. 10E) value of τ_{Σ} (dotted line in FIG. 10C and FIG. 10F) results in a difference of approximately 14% for the liquid phase and 9% for the solid phase, which is in good agreement with our model (Eq. (9)). It was noted that for the solid phase model, the variation between the donor and the acceptor is much smaller than for the liquid phase model; this is attributed to the smaller difference between the dynamic modulation data as shown in FIG. 10E.

To estimate how quickly the all-optical excitonic switch could in theory be cycled between the ON and OFF states, the absorbance cross section is required. The absorbance cross sections (σ) of the all-optical excitonic switch were determined for the photochromic nucleotides in either the open or closed configuration using τₒ and τ_{c}, and were found to be 7.7×10⁻²¹ and 1.4×10⁻²¹ m², (Eqs. 42-44), respectively, which align closely to values reported for non-nucleosidic diarylethenes. It should be noted that the values of τₒ, τ_{c}, τ_{Σ}, and tₛ determined from the time trial experiments are dependent upon the photon flux (2.3×10¹⁹ m⁻²·s⁻¹ (CLOSE) and 8.6×10¹⁹ m⁻²·s⁻¹ (OPEN) of the incoherent Xenon light source used for this work, see “Photon energy, photon fluence, and photon flux” section), and ultimately limited by the detector integration time (0.1 seconds here). Given these dependencies and the extracted absorbance cross sections, the ultrafast laser switching calculations above show it should be possible to obtain a tₛ in the picosecond range by using a high peak power ultrafast laser (i.e., greater photon flux and shorter exposure time) with a photon flux of approximately 10²⁰ to 10²¹ m⁻² per pulse. In fact, intrinsic cycle times on the order of picoseconds have been demonstrated for the opening and closing of non-nucleoside photochromic diarylethenes. As a frame of reference, this suggests the all optical switch has the potential to attain switching speeds similar to state-of-the-art transistors. (See Example 8, Table 8, and Table 9).

### Time Trial Design

For the time trial experiments, the photochromic nucleotides were exposed for cycle times (t) of 1 second, 3 seconds, 5 seconds, 10 seconds, 30 seconds, 100 seconds, 300 seconds, and 1000 seconds (Table 7). Each cycle of the time trial was performed as a series of four sequential excitation/emission scans whereby i) the all-optical excitonic switch was initially set to the ON state and a static emission scan (400 nm to 650 nm) was collected while exciting the donor of the all-optical excitonic switch with 350 nm VIS light, ii) the photochromic nucleotides were exposed to 300 nm UV light (τ_{c}) while simultaneously collecting donor emission (460 nm), iii) a static emission scan (400 nm to 650 nm) was collected while exciting the donor of the all-optical excitonic switch with 350 nm VIS light, and iv) the photochromic nucleotides were exposed to 455 nm VIS light (τₒ) while simultaneously collecting acceptor emission (520 nm). Note that fitting of very short time scales (1, 3, and 5 seconds) is questionable due to the difficulty of fitting segments too short to observe curvature.

TABLE 6
Summed exposure times (τ_{Σ}) for time trial experiments conducted with the
all-optical excitonic switch. One complete cycle (tₒ + t_{c}) is given as τ_{Σ} and the
average time of all values with the standard deviations are included.

| | | | | | | | | Averaged | |
|---|---|---|---|---|---|---|---|---|---|
| | | 5 | 10 | 30 | 100 | 300 | 1000 | time | Standard |
| | | sec | sec | sec | sec | sec | sec | (sec) | Deviation |
| LIQUID | tₒ | 5.44 | 5.07 | 5.68 | 5.93 | 6.14 | 5.75 | 5.67 | ±0.37 |
| 20 μM | t_{c} | 7.49 | 8.01 | 8.84 | 8.62 | 8.77 | 8.46 | 8.37 | ±0.52 |
| | τ_{Σ} | 12.93 | 13.08 | 14.52 | 14.55 | 14.91 | 14.21 | 14.04 | ±0.83 |
| SOLID | tₒ | 1.90 | 10.89 | 13.97 | 14.43 | 14.40 | 15.06 | 11.77 | ±5.06 |
| 69 mM | t_{c} | 2.84 | 7.44 | 9.47 | 9.49 | 9.67 | 9.96 | 8.14 | ±2.75 |
| | τ_{Σ} | 4.74 | 18.32 | 23.45 | 23.92 | 24.07 | 25.02 | 19.92 | ±7.81 |

### Example 7

### Cyclic Fatigue Assessment

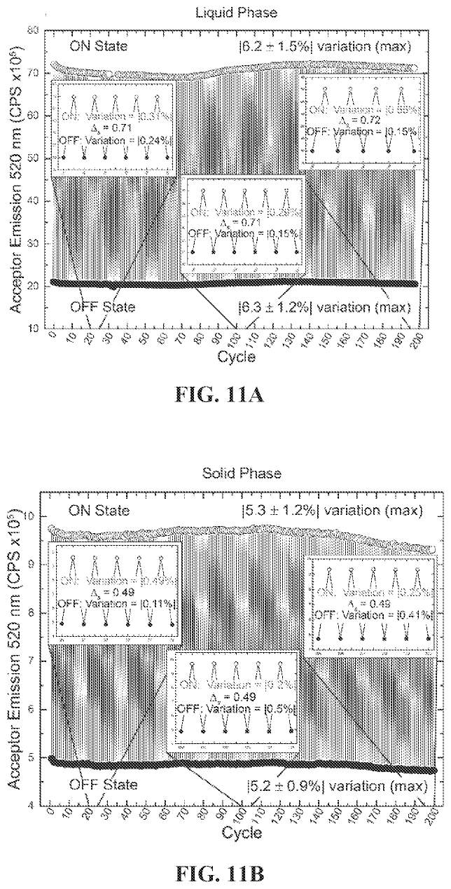

To move toward technologically relevant all-optical excitonic switches, prolonged cycling with minimal cyclic fatigue is required. To date, FRET-based optical switches and logic gates have shown, at most, twenty cycles, after which cyclic fatigue ensues. Cycling of the all-optical excitonic switch between ON and OFF states while monitoring the acceptor emission in both the liquid and solid phase was performed nearly 200 times as shown in FIG. 11A and FIG. 11B.

FIG. 11A-FIG. 11B show the cyclic fatigue assessment of an exemplary all-optical switch. FIG. 11A shows the cycling of the all-optical excitonic switch operated at a concentration of 20 μM in the liquid, and FIG. 11B shows the cycling of the all-optical excitonic switch operated at a concentration of 52 mM in the solid phases with an exposure time of 30 seconds performed nearly 200 times over the course of 100 minutes. Dynamic modulation scans were performed by exposing the photochromic nucleotides with ultraviolet (UV) λₑₓ^{ON→OFF} 300 nm or visible (VIS) λₑₓ^{OFF→ON} 455 nm light while monitoring the acceptor emission at 520 nm. Saw-tooth plots were created by averaging the final ten seconds of each cycling event to produce a single data point. About 6% or 5% random variation over the entire cycling range was determined for both the ON and OFF states in the liquid and the solid, respectively. Insets show that little cycle to cycle variation is observed, with values of both variation and Δₛ exhibiting little change. No apparent cyclic fatigue is observed over the course of the trial within the resolution of the experiment.

The maximum random variation for either the ON or OFF states is |6%| or less, most likely due to thermal drift of the spectrometer. To minimize the effect of spectrometer drift, the insets in FIG. 11A and FIG. 11B provide an alternate method to assess the variation in cycling by examining Δₛ and the variation over five cycles in three separate time regions. In these three regions, the variation is very small (≤0.55%). Additionally, comparing Δₛ and the variation within these three regions reveals values that are very similar, indicating no apparent evidence of cyclic fatigue. The absence of cyclic fatigue is most likely attributed to superior cyclic fatigue resistance of the diarylethene photochromic nucleotide as well as the photostability of the chromophore selection.

The results in this Example for the all-optical excitonic switch demonstrate exceptional cyclic fatigue resistance in the liquid and solid phases, as well as over two orders of magnitude greater cycling when compared to other DNA scaffolded FRET-based excitonic switches.

### Example 8

Comparison of the all-Optical Excitonic Switch to a 14 nm Node FinFET

Although a direct comparison of the size and speed of our all-optical excitonic switch to a state-of-the-art (SOTA) MOSFET is arguably not directly possible, a simple comparison provides some insight into physical parameters of interest. Here, the SOTA MOSFET used in the comparison is a bulk 14 nm technology node FinFET that appears to be used in Intel's 14 nm technology node computer processing units (CPUs), which includes Intel's cutting-edge Broadwell, Skylake, and Kaby Lake CPUs. While Intel uses a three-FinFET configuration in their 14 nm technology node, we will only compare one FinFET and neglect gate pitch for simplicity. With these assumptions, the FinFET parameters calculated here can be considered lower bounds. Parameters for the bulk 14 nm technology FinFET are shown in Table 7.

Both the power and energy of the FinFET can be estimated using the simple relationship,

P=C_{load}V_{dd}²f+IₗₑₐₖV_{dd}, (49)

E=P/f, (50)

where P, E, C_{load}, V_{dd}, f, and Iₗₑₐₖ denote power, energy, load capacitance, supply voltage, operation frequency, and leakage current of a transistor, respectively. If we assume that Iₗₑₐₖ is low (not necessarily the case, but this favors the FinFET in the comparison), we can ignore the I_{lead}V_{dd} term and Equation 49 becomes:

P=C_{load}V_{dd}²f. (51)

We can approximate the C_{load} as the capacitance, Cᵢₙᵥ, when the finFET is in inversion:

$${C}_{load} - {C}_{inv} = \frac{{L}_{g} {W}_{fin} {ɛ}_{0} {k}_{o x}}{E O T + {t}_{Q}} , \qquad ( 52 )$$

where L_{g}, W_{fin}, εₒ, kₒₓ, EOT, and t_{Q} are the gate length, fin width, permittivity of free space, high-k dielectric constant (HfO₂), effective oxide thickness and quantum mechanical inversion layer thickness⁷, respectively. EOT is given by:

$$E O T = {t}_{IL} + \frac{{k}_{IL}}{{k}_{H K}} {t}_{H K} . \qquad ( 53 )$$

where t_{IL}, t_{HK}, k_{IL}, and k_{HK} are the interfacial SiO₂ layer thickness, the interfacial SiO₂ layer relative dielectric constant, and the relative dielectric constant of the HfO₂, respectively. Using Equation 51, we can write the energy as:

E=C_{load}V_{dd}² (54)

For a FinFET, we can now calculate the values of C_{load}, E, and the energy for a full cycle (i.e., 2 E), which we define as ON-OFF-ON (see next paragraph). The values used for these calculations are listed in Table 7.

TABLE 7
Bulk Low Power 14 nm technology node FinFET parameters

| | |
|---|---|
| Gate Length (L_{g}): | 14 nm |
| Fin Width (W_{fin}): | 10 nm |
| Fin Height (H_{F}): | 100 nm |
| Field oxide Height (H_{FO}): | 300 nm |
| SiO₂ Interfacial Layer Relative | 3.9 |
| Dielectric Constant (k_{IL}): | |
| High-k Gate Oxide Thickness (t_{HK}): | ~1.25-2.2 nm (used 1.5 |
| | nm) calculation) |
| HfO₂ Relative Dielectric Constant (k_{HK}): | 25 |
| Interfacial SiO₂ layer thickness (t_{IL}) | ~1 nm |
| Quantum Mechanical Inversion Layer | 0.3-0.4 nm (used 0.4 nm) |
| Thickness (t_{Q}) | |
| Source or Drain Contact Pad Area Regions | L_{s,d} × L_{s,d} = 40 × 40 nm² |
| (A_{s,d}): | |
| Source/Drain Contact Pad Area | 15 nm |
| Regions − Distance from Gate (SD_{g}): | |
| Distance from Source to Drain (SD_{d}): | L_{g} + 2 × SD_{g} = 44 nm |
| Distance from End of Source to End of | SD_{d} + 2 × L_{s,d} = 124 nm |
| Drain (SDₜₒₜ): | |
| FinFET Footprint (A_{FET}) | L_{s,d} × SDₜₒₜ = 496 nm² |
| FinFET Volume (V_{FET}) | A_{FET} × H_{F} = 49,600 nm³ |
| Drain or power supply voltage (V_{dd}): | ~0.7-1.2 V (used 0.7 V in |
| | calculation) |
| Simulated Ring Oscillator Initial Cycle time⁸ | 10 s of ps |
| C_{FinFET}~Cᵢₙᵥ (Equation 52) | 0.059 fF |
| Energy/Cycle (Equation 54) | 58 aJ |

Though this approach is simplistic, the minimum energy requirements for switching the all-optical excitonic switch can be estimated using Equation 45 and multiplying the resultant photon energy values by the number of photochromic nucleotides present. For our device construct with three diarylethenes per photochromic nucleotide strand, the energy required to switch from the ON state to the OFF state (i.e., the open configuration to the closed configuration) is 1.98 aJ (i.e., 3 times the 0.66 aJ energy of a single 300 nm photon). The energy required for the reverse process (i.e., OFF to ON state or closed to open configuration) is less, 1.32 aJ, due to the lower energy per photon of the 455 nm photons used. Thus, the total energy consumed in one full switching cycle (i.e., ON-OFF-ON or OFF-ON-OFF) is 3.3 aJ. Full parameters for the all-optical excitonic switch are shown in Table 8. Times to open and close the diarylethene photochromic nucleotide are taken from references.

TABLE 8
Parameters for All-Optical Excitonic Switch

| | |
|---|---|
| Length (L): | ~20 bp × 0.34 nm = 6.8 nm |
| Width (W): | 2 nm |
| Height (H): | 2 nm + 1 nm = 3 nm |
| Footprint Area (A): | L × W = 13.6 nm² |
| Volume (V): | A × H = 40.8 nm³ |
| Diarylethene decay time (t_{d}) | 4-40 ps |
| Diarylethene open time (tₒ) | 40 ps |
| Diarylethene close time (t_{c}) | 4 ps |
| Cycle time (t_{c}) | tₒ + t_{c} = 44 ps |
| Energy/Cycle | 3.3 aJ |

The energy to flip the FinFET to the ON state (i.e., into strong inversion), using equation 54, was determined to be ˜29 aJ. The energy to flip the FinFET to the OFF state is assumed to be the same, hence the energy per cycle (i.e., flipping from OFF to ON to OFF states) was calculated to be 58 aJ. Note that this energy/cycle is a lower bound since the energy due to the leakage current in the OFF state is ignored (see equations 49 and 50 and accompanying assumptions). Comparisons between similar parameters for the FinFET (Table 7) and the all-optical excitonic switch (Table 8) reveal that the all-optical switch's footprint is 37× more compact, volume is over 3 orders of magnitude smaller, and energy consumption is over an order of magnitude less than that of the 14 nm FinFET. While it is difficult to find directly measured finFET cycle times, simulations show 14 nm FinFET ring oscillator initial cycle times in the 10 s of ps and simulated write times to 6-finFET static random access memory (SRAM) in the 1 s to 10 s of ps, but do not discuss read times. It is reasonable to assume that cycle times for a single finFET is in the 10 s of ps which is certainly comparable to or faster than the photoisomerization reaction cycle times for diarylethene molecules. Although the calculations we have employed are simple first order approximations that one can argue should not be used as direct comparisons, they are rather compelling and do allude to the potential capabilities and impact of the all-optical excitonic switch for both low power and high speed applications relative to the current semiconductor electronics state of the art. Finally, in addition to the lower energy required per switching cycle for the all-optical excitonic switch calculated in the above comparison, it should be noted that the FinFET supply voltage, V_{dd}, is always applied, whereas once the all optical excitonic switch is in a given state (ON or OFF), the state is stable and thus no additional energy is required to maintain that state.

### Example 9

### Determination of the Switching Efficiency of PS1 (Quantitative Composition of the Photostationary State)

High-performance liquid chromatography (HPLC) was conducted in order to determine the maximal switching efficiency (i.e., the photostationary state composition) of single photochromic nucleotide strands.

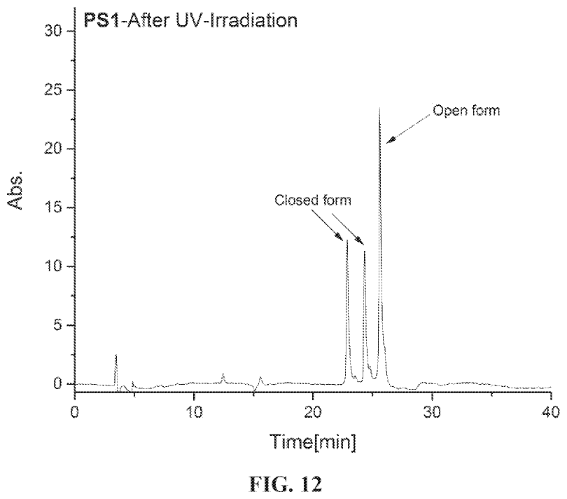

The switching efficiency was determined by irradiating PS1 for 10 min with a 310 nm LED in a 50 μL cuvette with a concentration of 3 μM. The sample was then analyzed via an HPLC with a Synergi Fusion-RP (80 Å, 4 μm, 150×30 mm, Flowrate 1 mL/min) to separate the two different isomers. The HPLC method D is listed in Table 9. The mobile phases were Buffer A (0.1 M triethylammonium acetate in water, pH 7) and Buffer B (0.1 M triethylammonium acetate in 80% acetonitrile). FIG. 12 shows the HPLC time trace of PS1 after 10 min of UV-irradiation with a 310 nm LED (Thorlabs M310L3) at 260 nm.

The two diastereomers of the closed ring form also absorb light in the visible range and can therefore be differentiated from the open isomer. Integration of the corresponding peaks leads to the ratio between the open and the closed isomer. The extinction coefficients of both isomers at 260 nm are nearly the same due to the strong absorption of the nucleotides at this wavelength, which allows the integration of the peaks to extract the switching efficiency.

Separation and identification of the two possible closed-ring isomers and one open-ring isomer in the HPLC data indicates a switching efficiency of 50±2% from the open to the closed form at the photostationary state and near unity conversion to the open form (<0.2% closed form, below detectable limit) for closed to open switching.

TABLE 9
HPLC gradient for the determination of the switching efficiency of PS1
Method D

| Time [min] | Buffer B [%] |
|---|---|
| 0 | 7.5 |
| 10 | 12.5 |
| 35 | 40 |
| 40 | 50 |
| 45 | 100 |
| 50 | 100 |
| 52 | 7.5 |

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The inventions being thus described, it will be obvious that the same may be varied in many ways. Such variations are not to be regarded as a departure from the spirit and scope of the inventions and all such modifications are intended to be included within the scope of the following claims.

1. An all-optical excitonic switch comprising:
an oligonucleotide;
a donor chromophore having spectral overlap with an acceptor chromophore and positioned for excitonic transfer; and
a photochromic nucleotide which is capable of undergoing reversible photoisomerization.

2. The switch according to claim 1 comprising the donor chromophore, acceptor chromophore, and the photochromic nucleotide positioned on a single oligonucleotide.

3. The switch according to claim 1 comprising the donor chromophore, acceptor chromophore, and the photochromic nucleotide positioned on two or more oligonucleotides which form a duplex.

4. The switch according to claim 1, wherein the oligonucleotides is made of RNA, DNA, PLA, LNA, BNA, or combinations thereof.

5. The switch according to claim 1, wherein the photochromic nucleotide is a modified nucleotide having a modified nitrogenous base.

6. The switch according to claim 1, wherein the photochromic nucleotide has a modified adenine, guanine, cytosine, thymine, or uracil base.

7. The switch according to claim 1, wherein the photochromic nucleotide has a modified uracil or cytosine base.

8. The switch according to claim 1, wherein the photochromic nucleotide comprises a diarylethene group.

9. The switch according to claim 1, wherein the photochromic nucleotide comprises an azobenzene group.

10. The switch according to claim 1, wherein the photochromic nucleotide has a base comprising a 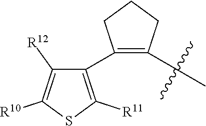
group, where R¹⁰ and R¹² are independently H, CH₃, an alkyl, or together form a ring; and R¹¹ is H, CH₃, or an alkyl.

11. The switch according to claim 10, wherein the photochromic nucleotide has a base of 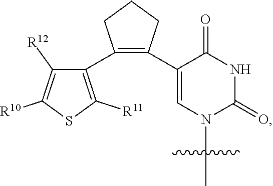
where R¹⁰ and R¹² are independently H, CH₃, an alkyl, or together form a ring; and R¹¹ is H, CH₃, or an alkyl.

12. The switch according to claim 11, wherein:
(i) R¹⁰ is aryl group and R¹¹ and R¹² are independently H, CH₃, or alkyl;
(ii) R¹⁰ and R¹² are together a C₅ or C₆ member ring and R¹¹ is H, CH₃, or alkyl;
(iii) R¹⁰ and R¹² are together a substituted or unsubstituted aromatic ring and R¹¹ is H, CH₃, or alkyl;
(iv) R¹⁰ is aryl group; R¹¹ is CH₃; and R¹² is H;
(iv) R¹⁰ is —C₆H₅; R¹¹ is CH₃; and R¹² is H; or
(vi) R¹⁰ is a substituted aryl group; R¹¹ is CH₃; and R¹² is H.

13-17. (canceled)

18. The switch according to claim 1, comprising:
(i) two identical or different photochromic nucleotides;
(ii) three, identical or different, or a combination thereof, photochromic nucleotides; or
(iii) four or more, identical or different, or a combination thereof, photochromic nucleotides.

19-20. (canceled)

21. The switch according to claim 1, where the photochromic nucleotides are:
(i) positioned on the same oligonucleotide; and/or
(ii) dU^{ps}, defined by the formula:

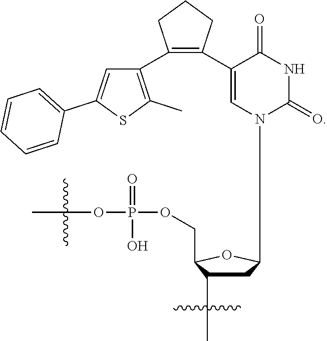

22. (canceled)

23. The switch according to claim 1, wherein the oligonucleotide comprises:
(i) a CXA CXA or CXA CXA CXA sequence, where X is a photochromic nucleotide; and/or
(ii) a CdU^{ps} A CdU^{ps} A or CdU^{ps} A CdU^{ps} A CdU^{ps} A sequence; and/or
(iii) a GGC TAG CGA CdU^{ps} ACdU^{ps} A or GGC TAG CdU^{ps} A CdU^{ps} A CdU^{ps} A sequence.

24-25. (canceled)

26. The switch according to claim 1, wherein:
(i) the donor is A390;
(ii) the acceptor is A488; and
(iii) the photochromic nucleotide is dU^{ps}.

27-28. (canceled)

29. The switch according to claim 26, wherein:
(i) the oligonucleotide comprises a donor-AGT AGT AGC TAG CCG CAC GCA CCG GCT CG sequence; and
(ii) the oligonucleotide comprises a CGA GCC GGT GCG TGC-acceptor sequence.

30. (canceled)

31. The switch according to claim 26, wherein:
(i) the donor has an emission band of from about 400 nm to about 550 nm;
(ii) the acceptor has absorption band of from about 400 nm to about 550 nm; and
(iii) the photochromic nucleotides have an absorption band of from about 370 nm to about 570 nm.

32-33. (canceled)

34. The switch according to claim 1, wherein the switch can operate in solid state and in liquid state.

35. (canceled)

36. A device comprising one or more all-optical excitonic switches according to claim 1, wherein the one or more all-optical excitonic switches function as memory or transistors.


SEQUENCE LISTING (6 sequences)

SEQ ID NO: 1 - DNA, 29 bp, Artificial Sequence
  Donor sequence
agtagtagctagccgcacgcaccggctcg

SEQ ID NO: 2 - DNA, 15 bp, Artificial Sequence
  Accecptor Sequence
cgagccggtgcgtgc

SEQ ID NO: 3 - DNA, 15 bp, Artificial Sequence
  Photochromatic sequence with 1 photochromatic nucleotide
  modified_base (11)..(11): deoxyuridine-based photoswitchable nucleoside with phenyl substituent (dUps)
ggctagctacuacga

SEQ ID NO: 4 - DNA, 15 bp, Artificial Sequence
  Phtochromatic sequence with 3 phtochromatic nucleotides
  modified_base (8)..(8): deoxyuridine-based photoswitchable nucleoside with phenyl substituent (dUps)
  modified_base (11)..(11): deoxyuridine-based photoswitchable nucleoside with phenyl substituent (dUps)
  modified_base (14)..(14): deoxyuridine-based photoswitchable nucleoside with phenyl substituent (dUps)
ggctagcuacuacua

SEQ ID NO: 5 - DNA, 15 bp, Artificial Sequence
  Photochromatic control sequence
ggctagctactacta

SEQ ID NO: 6 - DNA, 15 bp, Artificial Sequence
  Phtochromatic sequence with 2 photochromatic nucleotides
  modified_base (11)..(11): deoxyuridine-based photoswitchable nucleoside with phenyl substituent (dUps)
  modified_base (14)..(14): deoxyuridine-based photoswitchable nucleoside with phenyl substituent (dUps)
ggctagcgacuacua